(19) United States
(12) Patent Application Publication
(10) Pub. No.: US 2025/0318755 A1
(43) Pub. Date: Oct. 16, 2025

(54) TECHNIQUES FOR EVALUATING IMPACTS BETWEEN PHYSIOLOGICAL DATA AND METABOLIC HEALTH

(71) Applicant: Oura Health Oy, Oulu (FI)

(72) Inventors: Heli Tuulia Koskimäki, Oulu (FI); Pauli Juhani Ohukainen, Oulu (FI); Veera Lindholm, Oulu (FI); Henri Matti Johannes Huttunen, Pudasjärvi (FI); Antti Matias Hyytinen, Oulu (FI)

(21) Appl. No.: 19/174,812

(22) Filed: Apr. 9, 2025

Related U.S. Application Data
(60) Provisional application No. 63/632,849, filed on Apr. 11, 2024.
(60) Provisional application No. 63/656,587, filed on Jun. 5, 2024.

(51) Int. Cl. A61B 5/145; A61B 5/00; A61B 5/1455

(52) U.S. Cl. CPC A61B 5/14532; A61B 5/0002; A61B 5/1455; A61B 5/4809; A61B 5/6826; A61B 5/7264

(57) ABSTRACT

Methods, systems, and devices for evaluating a user's metabolic balance are described. A system may include a wearable device and a glucose monitoring device. The system may acquire physiological data from the wearable device, and blood glucose data from the glucose monitoring device. The system may be configured to determine physiological characteristics of the user based on the physiological data, such characteristics associated with a sleep quality of the user, a stress level of the user, an activity level of the user, a recovery of the user, or any combination thereof. Similarly, the system may identify metabolic characteristics of the user, such as changes in the user's blood glucose data. By overlaying and comparing the physiological data and the blood glucose data, the system may be able to evaluate how the user's physiological characteristics affect or impact the user's metabolic characteristics, or vice versa.

### CROSS REFERENCE

The present Application for Patent claims the benefit of U.S. Provisional Patent Application No. 63/656,587 by KOSKIMÄKI et al., entitled “TECHNIQUES FOR EVALUATING IMPACTS BETWEEN PHYSIOLOGICAL DATA AND METABOLIC HEALTH,” filed Jun. 5, 2024, and U.S. Provisional Patent Application No. 63/632,849 by KOSKIMÄKI et al., entitled “TECHNIQUES FOR EVALUATING IMPACTS BETWEEN PHYSIOLOGICAL DATA AND METABOLIC HEALTH,” filed Apr. 11, 2024, both of which assigned to the assignee thereof, and expressly incorporated by reference herein.

### FIELD OF TECHNOLOGY

The following relates to wearable devices and data processing, including techniques for evaluating impacts between physiological data and metabolic health.

### BACKGROUND

Some glucose monitoring devices may be able to collect blood glucose data from a user, and provide the user with alerts or other information regarding their blood glucose levels and overall metabolic health. However, current glucose monitoring devices may be unable to provide additional context regarding why the user's blood glucose levels fluctuate the way they do, and may be otherwise unable to provide additional context regarding the user's overall metabolic health.

### BRIEF DESCRIPTION OF THE DRAWINGS

FIG. 1 illustrates an example of a system that supports techniques for evaluating impacts between physiological data and metabolic health in accordance with aspects of the present disclosure.

FIG. 2 illustrates an example of a system that supports techniques for evaluating impacts between physiological data and metabolic health in accordance with aspects of the present disclosure.

FIG. 3 shows an example of a system that supports techniques for evaluating impacts between physiological data and metabolic health in accordance with aspects of the present disclosure.

FIG. 4 shows an example of a sleep diagram that supports techniques for evaluating impacts between physiological data and metabolic health in accordance with aspects of the present disclosure.

FIG. 5 shows an example of an activity diagram that supports techniques for evaluating impacts between physiological data and metabolic health in accordance with aspects of the present disclosure.

FIG. 6 shows an example of a meal timing diagram that supports techniques for evaluating impacts between physiological data and metabolic health in accordance with aspects of the present disclosure.

FIG. 7 shows examples of glucose diagrams that support techniques for evaluating impacts between physiological data and metabolic health in accordance with aspects of the present disclosure.

FIG. 8 shows an example of graphical user interfaces (GUIs) that support techniques for evaluating impacts between physiological data and metabolic health in accordance with aspects of the present disclosure.

FIG. 9 shows a block diagram of an apparatus that supports techniques for evaluating impacts between physiological data and metabolic health in accordance with aspects of the present disclosure.

FIG. 10 shows a block diagram of a wearable application that supports techniques for evaluating impacts between physiological data and metabolic health in accordance with aspects of the present disclosure.

FIG. 11 shows a diagram of a system including a device that supports techniques for evaluating impacts between physiological data and metabolic health in accordance with aspects of the present disclosure.

FIGS. 12 and 13 show flowcharts illustrating methods that support techniques for evaluating impacts between physiological data and metabolic health in accordance with aspects of the present disclosure.

### DETAILED DESCRIPTION

Some glucose monitoring devices may be able to collect blood glucose data from a user, and provide the user with alerts or other information regarding their blood glucose levels and overall metabolic health. However, current glucose monitoring devices may be unable to provide additional context regarding why the user's blood glucose levels fluctuate the way they do, and may be otherwise unable to provide additional context regarding the user's overall metabolic health. In other words, conventional glucose monitoring devices may be able to tell the user what their blood glucose levels are, but may be unable to tell the user why their blood glucose levels are where they are.

Some users keep a food log of all the things they eat and drink, and cross-reference the food log with blood glucose data collected via a glucose monitoring device to gain additional insights into their metabolic health. However, such meal tracking techniques may be time consuming and burdensome. Moreover, even if a user keeps track of all the food and drink they put into their body, such information does not tell the whole story regarding their metabolic health. In particular, food tracking techniques may not take into account other physiological characteristics that may affect the user's blood glucose and overall metabolic health, such as stress levels, sleep quality, activity patterns, and the like.

Accordingly, aspects of the present disclosure are directed to techniques for evaluating how a user's physiological characteristics (e.g., sleeping patterns, eating patterns, exercise patterns, stress levels, etc.) affect the user's blood glucose levels and overall metabolic health. Conversely, other aspects of the present disclosure are directed to techniques for evaluating how a user's blood glucose levels and metabolic characteristics affect other aspects of the user's health and physiological data, such as sleep quality, stress levels, etc. In this regard, aspects of the present disclosure may provide a mechanism for evaluating both blood glucose data and other physiological data to provide more actionable insights into the users physiological and metabolic health. As such, techniques described herein may provide a more holistic approach to evaluating the user's physiological health, which may enable the user to make actionable changes in their lives to improve their health.

Stated differently, aspects of the present disclosure move beyond simple food tracking techniques to foster a holistic approach for evaluating and improving the overall well-being and health span of users. Techniques described may determine information regarding the impact of their food habits, sleep patterns, stress, body composition, and more. Techniques described herein may provide users with more information, such as alerts, regarding the impact of their food habits, sleep patterns, stress, body composition, and more. In doing so, aspects of the present disclosure may empower users to make more informed choices for their metabolic health, weight management, and chronic disease prevention, while nurturing a psychologically healthy relationship with food.

Aspects of the disclosure are initially described in the context of systems supporting physiological data collection from users via wearable devices. Additional aspects of the disclosure are described in the context of an example sleep diagram, an example activity diagram, an example meal timing diagram, and example glucose diagrams. Aspects of the disclosure are further illustrated by and described with reference to apparatus diagrams, system diagrams, and flowcharts that relate to techniques for evaluating impacts between physiological data and metabolic health.

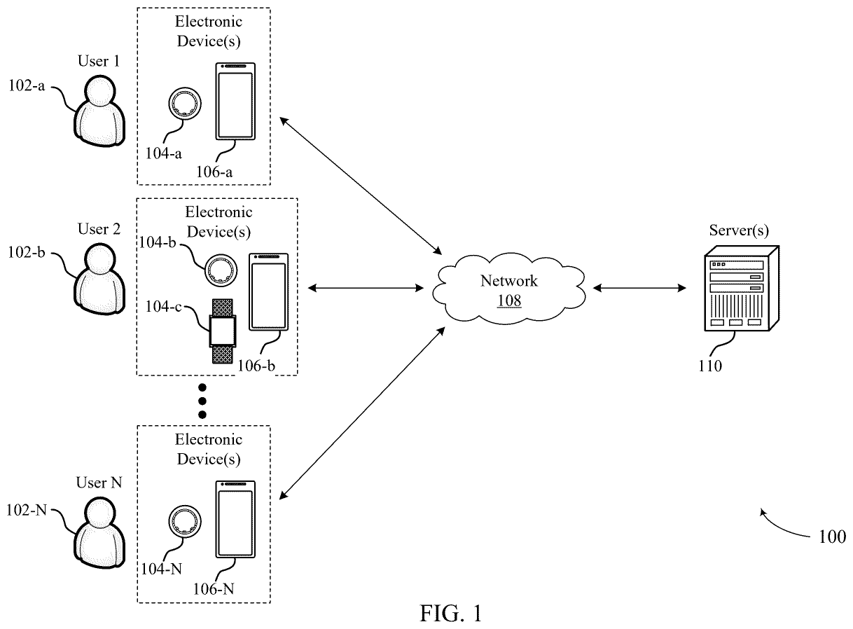

FIG. 1 illustrates an example of a system 100 that supports techniques for evaluating impacts between physiological data and metabolic health in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for evaluating how a user's physiological characteristics (e.g., sleeping patterns, eating patterns, exercise patterns, stress levels, etc.) affect the user's blood glucose levels and overall metabolic health. Conversely, other aspects of the present disclosure are directed to techniques for evaluating how a user's blood glucose levels and metabolic characteristics affect other aspects of the user's health and physiological data, such as sleep quality, stress levels, etc. For example, the system 100 may be configured to determine how a user's blood glucose data affects their Sleep Score, Readiness Score, Activity Score, Stress Score, or any combination thereof.

In this regard, aspects of the present disclosure may provide a mechanism for evaluating both blood glucose data and other physiological data to provide more actionable insights into the users physiological and metabolic health. As such, techniques described herein may provide a more holistic approach to evaluating the user's physiological health, which may enable the user to make actionable changes in their lives to improve their health.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

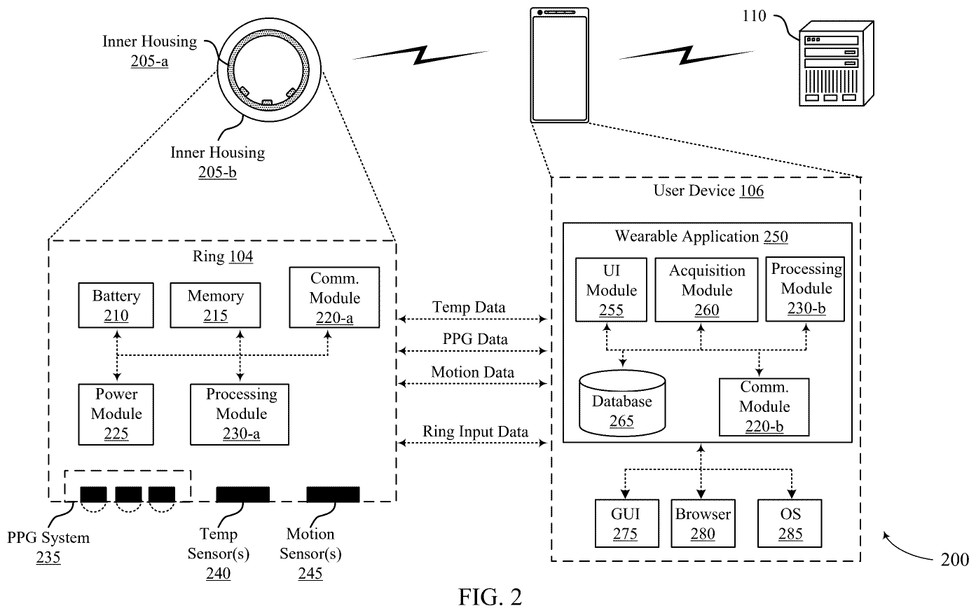

FIG. 2 illustrates an example of a system 200 that supports techniques for evaluating impacts between physiological data and metabolic health in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-acommunicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, data collected by the wearable device 104, and/or analyses performed by the wearable device 104, the user device 106, and/or the servers 110, may be used to adjust operational parameters of the wearable device 104. For example, based on a determined heart rate of the user and/or a determined activity state of the user, the wearable device 104 may adjust a sampling rate for measuring the user's heart rate, and/or may activate or deactivate certain sensors and/or physiological measurements (e.g., deactivate SpO2 measurements when the user is engaged in physical activity, or otherwise exhibits an activity/movement level above some threshold). By way of another example, the user device 106 and/or the servers 110 may calculate a Readiness Score for the user, and may deactivate or disable activity measurements performed by the wearable device 104 in cases where the Readiness Score is below some threshold (in order to reduce power consumption and conserve battery at the wearable device 104, and/or to disincentivize the user from performing rigorous activity when their Readiness Score is below the threshold value). In this regard, any measurements, calculations, and/or analyses performed by the various devices within the system 100 (e.g., wearable device 104, user device 106, servers 110) may be used by the system 100 to control and/or adjust the operational parameters of the wearable device 104.

Operational parameters that may be controlled/adjusted at the wearable device 204 based on collected data and/or analyses performed by the system 100 may include, but are not limited to, a periodicity/frequency that measurements are performed (e.g., sampling rate), a power level or intensity of LEDs, algorithms used to analyze data at the wearable device 104, what types of measurements are performed (e.g., enabling/disabling specific sensors or types of measurements), a periodicity or frequency that the wearable device 104 transmits data to the user device 106, or any combination thereof. Adjusting operational parameters of the wearable device 104 based on collected data and/or analyses performed by the system 200 may reduce power consumption and improve battery performance at the wearable device 104, and may lead to higher quality data collected by the wearable device 104, thereby enabling the system 200 to perform more accurate and reliable analyses/diagnoses of the user's physiological parameters, and leading to better guidance and insights that may enable the user to improve their overall health.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the respective devices of the system 200 may support techniques for evaluating how a user's physiological characteristics (e.g., sleeping patterns, eating patterns, exercise patterns, stress levels, etc.) affect the user's blood glucose levels and overall metabolic health. Conversely, other aspects of the present disclosure are directed to techniques for evaluating how a user's blood glucose levels and metabolic characteristics affect other aspects of the user's health and physiological data, such as sleep quality, stress levels, etc. For example, the system 100 may be configured to determine how a user's blood glucose data affects their Sleep Score, Readiness Score, Activity Score, Stress Score, or any combination thereof.

In this regard, aspects of the present disclosure may provide a mechanism for evaluating both blood glucose data and other physiological data to provide more actionable insights into the users physiological and metabolic health. As such, techniques described herein may provide a more holistic approach to evaluating the user's physiological health, which may enable the user to make actionable changes in their lives to improve their health.

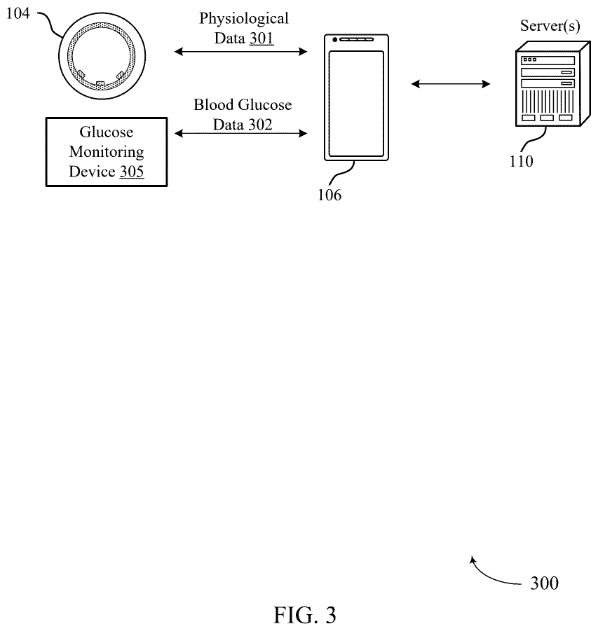

FIG. 3 shows an example of a system 300 that supports techniques for evaluating impacts between physiological data and metabolic health in accordance with aspects of the present disclosure. Aspects of the system 300 may implement, or be implemented by, aspects of the system 100, the system 200, or both.

The system 300 may include a wearable device 104 (e.g., wearable ring device 104, wrist-worn wearable device 104, chest-worn wearable device 104, etc.), a glucose monitoring device 305, a user device 106, and one or more servers 110, which may be examples of corresponding devices described herein. In some aspects, the wearable device 104 may be configured to acquire physiological data 301 from a user, such as PPG data, motion data, temperature data, SpO2 data, or any combination thereof. Similarly, the glucose monitoring device 305 may be configured to acquire blood glucose data 302 from the user.

The glucose monitoring device 305 may be an example of a biosensor coupled to the user. For example, the glucose monitoring device 305 may be attached to the arm of the user, a stomach of the user, a hip of the user, a leg of the user, or a combination thereof.

In some implementations, the wearable device 104 and the glucose monitoring device 305 may include separate devices. In other cases, the wearable device 104 and the glucose monitoring device 305 may be included within the same device. For example, in some implementations, the glucose monitoring device 305 may be implemented as a component or module of the wearable device 104.

For instance, in some implementations, glucose monitoring device 305 may be implemented as a component or module of the wearable device 104 (e.g., either built into the wearable ring device 104, or attached to the wearable ring device 104 such as via an adhesive or other means). For instance, in some aspects, the glucose monitoring device 305 may be configured as a disposable patch or module that can be attached to the inner curved surface of the wearable ring device 104 to contact a tissue of the user. In such cases, the glucose monitoring device 305 may be configured to acquire a sample of blood from the user (e.g., via microneedles or other means), where the blood sample may be analyzed using the PPG sensors (e.g., LEDs, photodetectors) of the wearable device 104. That is, in some cases, the sensors of the wearable device 104 may be configured to acquire both the physiological data 301 and the blood glucose data 302. In other cases, the wearable device 104 may have separate sets of LEDs and/or photodetectors that are dedicated for PPG-related measurements (e.g., physiological data 301) and blood glucose measurements (e.g., blood glucose data 302).

For instance, the glucose monitoring device 305 may include microfluidic channels that direct the blood sample to/in front of the LEDs and/or photodetectors so that the wearable ring device 104 may use light-based measurement techniques described herein to collect the blood glucose data 302. Additionally, or alternatively, the glucose monitoring device 305 may direct the blood sample (e.g., via microfluidic channels) to a test strip that is configured to change color based on the concentration of glucose in the blood sample (e.g., enzymes on the test strip change color in response to glucose). In this example, the LEDs and photodetectors of the wearable device 104 may be configured to detect the color change of the test strip, and may therefore determine the blood glucose level (e.g., blood glucose data 302) based on the color change. That is, the LEDs and photodetectors of the wearable device 104 may measure an initial or baseline color of the test strip prior to the blood sample (by measuring light that is reflected/absorbed by the test strip), and may measure the color change of the test strip (which is indicative of the blood glucose level) by performing additional measurements after the blood sample has been absorbed/contacted by the test strip.

In other cases, the wearable device 104 and/or the glucose monitoring device 305 may use electrochemical detection techniques to collect the blood glucose data 302. For example, electrodes of the glucose monitoring device 305 (and/or electrodes of the wearable device 104, such as electrodes on an inner curved surface of the wearable device) may measure an electrical signal generated by an enzymatic reaction between the glucose within a blood sample and enzymes on/within the glucose monitoring device 305 (e.g., enzymes of a test strip). In such cases, the measured electrical signal may be associated with (e.g., indicative of) the amount or concentration of glucose within the blood sample.

In cases where the glucose monitoring device 305 is connected to the wearable device 104 (e.g., affixed, attached, or implemented as a component of the wearable device 104), the glucose monitoring device 305 may leverage other components of the wearable device 305 to facilitate collection of the blood glucose data 302. For example, the glucose monitoring device 305 may or may not include its own battery, and may therefore use power received from a battery of the wearable device 104 in order to collect the blood glucose data 302.

In some other cases, the glucose monitoring device 302 may include its own processing and/or communication circuitry, and may be configured to communicate the blood glucose data 302 directly to the user device 106 and/or the wearable ring device 104. For instance, in some cases, the glucose monitoring device 305 may interface with or otherwise communicate with the wearable ring device 104 to transmit the blood glucose data 302 to the wearable ring device 104, where the blood glucose data 302 can be processed by the wearable device 104 and/or relayed to the user device 106 and/or servers 110 for processing.

In some aspects, the user device 106, the servers 110, or both, may be configured to acquire the physiological data 301 and the blood glucose data 302 from the wearable device 104 and the glucose monitoring device 305, respectively. In some aspects, the user device 106, the servers 110, or both, may be configured to evaluate how a user's physiological characteristics (e.g., sleeping patterns, eating patterns, exercise patterns, stress levels, etc.) affect the user's blood glucose levels and overall metabolic health (e.g., how the physiological data 301 affects the blood glucose data 302). For example, the user device 106, the servers 110, or both, may be configured to determine how the user's activity or sleeping patterns affect their blood glucose levels.

Conversely, user device 106, the servers 110, or both, may be configured to evaluate how a user's blood glucose levels and metabolic characteristics affect other aspects of the user's health and physiological data, such as sleep quality, stress levels, etc. (e.g., how the blood glucose data 302 affects the physiological data 301). For example, the user device 106, the servers 110, or both, may be configured to determine how a user's blood glucose data affects their Sleep Score, Readiness Score, Activity Score, Stress Score, or any combination thereof.

In this regard, aspects of the present disclosure may provide a mechanism for evaluating both blood glucose data 302 and other physiological data 301 to provide more actionable insights into the users physiological and metabolic health. As such, techniques described herein may provide a more holistic approach to evaluating the user's physiological health, which may enable the user to make actionable changes in their lives to improve their health.

In some aspects, the system 300 (e.g., user device 106, servers 110, etc.) may acquire physiological data 301 collected from a user via the wearable device 104. The physiological data 301 may include PPG data, motion data, temperature data, SpO2 data, and the like. The system 300 may determine one or more physiological characteristics of the user based on the physiological data 301. The one or more physiological characteristics may be associated with a sleep quality of the user (e.g., Sleep Score), a stress level of the user (e.g., Stress Score), an activity level of the user (e.g., Activity Score), a recovery of the user (e.g., Readiness Score), or any combination thereof. The system 300 may further acquire blood glucose data 302 collected from the user via the glucose monitoring device 305 and identify one or more impacts that the one or more physiological characteristics had on the blood glucose data 302. Identifying one or more impacts may be in one aspect be determining relationships between two or more variables and relating the relationships to one or more further variables. Subsequently, the system 300 may cause the user device 106 to convey information to the user associated with the one or more impacts. In other words, the user device 106 may indicate how the user's physiological data 301 has affected the blood glucose data 302. Conveying information may include a range of interactions with the user including alerts, physical, visual or audio stimuli, feedback, instructions, or information. The wearable device and/or user device calculates or predicts the physical state of the user, thus making a technical contribution regardless of what use is made of the results.

In additional or alternative implementations, the user device 106, the servers 110, or both, may be configured to determine one or more metabolic characteristics of the user based on the blood glucose data 302, and identify one or more impacts that the one or more metabolic characteristics had on the one or more physiological characteristics of the user. Subsequently, the system 300 may cause the user device 106 to convey information to the user associated with the one or more impacts. In other words, the user device 106 may indicate how the user's blood glucose data 302 has affected the user's physiological data 301. Moreover, the system 300 may be configured to generate alerts based on the user's blood glucose data 302 (e.g., trigger an alert when the user's blood glucose data 302 satisfies some threshold, such as a high blood glucose threshold or a low blood glucose threshold).

In some aspects, by identifying impacts of the physiological data 301 on the blood glucose data 302 (and vice versa), the system 300 may be able to more accurately identify medical conditions associated with the user's blood glucose data 302 and metabolic health (as compared to systems that analyze the user's blood glucose data 302 alone). Moreover, by identifying impacts of the physiological data 301 on the blood glucose data 302 (and vice versa), the system 300 may enable earlier identification of medical conditions associated with the user's blood glucose data 302 and metabolic health (as compared to systems that analyze the user's blood glucose data 302 alone). For instance, the system 300 may be configured to “learn” specific physiological characteristics and/or patterns between physiological characteristics that typically precede increases/decreases in the user's blood glucose data 302, and may therefore be able to alert the user of such likely changes in their blood glucose data 302 before such changes actually occur. For instance, the system 300 may be configured to “learn” that the user's respiration rate increases and their HRV decreases some time before the user's blood glucose levels fall. As such, upon identifying an increase in the user's respiration rate and a decrease in the user's HRV, the system 300 may trigger an alert to notify the user that their blood glucose levels are likely to fall in the near future (and/or trigger the system 300 to take actions to increase the user's blood glucose levels to prevent such a decrease, such as activating an insulin pump).

In some aspects, the user device 106, the servers 110, or both, may input the physiological data 301, the one or more physiological characteristics, and the blood glucose data 302 into a machine learning classifier, where the machine learning classifier may be configured to output an indication of the one or more impacts. In some cases, the one or more physiological characteristics may include a sleep debt of the user, and the one or more impacts may include an increase in the blood glucose data 302 of the user.

In some aspects, the user device 106, the servers 110, or both, may identify an eating gesture, a drinking gesture, or both, based on the motion data within the physiological data 301, where identifying the one or more impacts may be based on identifying the eating gesture, the drinking gesture, or both.

In some aspects, the user device 106, the servers 110, or both, may identify one or more changes in the blood glucose data 302, and identify an activity pattern of the user during a time interval preceding the one or more changes in the blood glucose data 302, where the one or more impacts may be based on a temporal relationship between the activity pattern and the one or more changes. As described previously herein, the temporal relationship may be used for early detection of medical conditions associated with the user's blood glucose data 302/metabolic health. For instance, the system 300 may “learn” specific activity patterns that typically precede a change in the user's blood glucose data 302, and may trigger alerts to notify the user of potential future changes in their blood glucose data 302.

In some aspects, the user device 106, the servers 110, or both, may identify a sleep period that the user may be asleep based on the physiological data 301, and may identify one or more meals consumed by the user based on the physiological data 301, the blood glucose data 302, or both, where the one or more impacts may be based on a temporal relationship between the sleep period and the one or more meals.

In some implementations, the system 300 may be configured to control or adjust operational parameters of one or more components of the system 300 based on the collected physiological data 301, the blood glucose data 302, based on identified impacts on the physiological data 301 on the blood glucose data 302 (or vice versa), or any combination thereof. In particular, the system 300 may adjust what types of measurements are performed, how measurements are performed, and/or how frequently measurements are performed, based on the identified impacts in order to better monitor the user's overall health, and trigger alerts of potentially dangerous conditions (e.g., high/low blood glucose levels). Further, selectively adjusting operational parameters of the respective devices may lead to improved power consumption and battery performance at the respective devices (e.g., by using a lower measurement periodicity until an identified physiological condition or impact triggers a higher measurement periodicity).

For example, upon identifying an impact of the physiological data 301 on the blood glucose data 302 (or vice versa), the user device 106, the servers 110, or both, may transmit a signal to the wearable device 104 and/or the glucose monitoring device 305 to adjust one or more operational parameters of the respective devices based on the identified impact(s). In other cases where the wearable device 104 and/or the glucose monitoring device 305 identify the impact(s) themselves, the devices may be configured to autonomously adjust the operational parameters (e.g., without an explicit signal from the user device 106 and/or servers 110). Operational parameters of the respective devices (e.g., wearable device 104, glucose monitoring device 305) that may be controlled or adjusted may include a periodicity/frequency with which measurements are performed (e.g., increase periodicity for performing blood glucose measurements), what types of measurements are performed (e.g., trigger the wearable device 104 to perform blood oxygen measurements), parameters for performing measurements (e.g., adjusting an LED intensity used to perform PPG measurements), or any combination thereof.

Further, in cases where the system 300 includes medical devices that are configured to adjust or control the user's blood glucose levels (e.g., an insulin pump), the system 300 may be configured to selectively control or adjust such devices based on the collected physiological data 301, the blood glucose data 302, based on identified impacts on the physiological data 301 on the blood glucose data 302 (or vice versa), or any combination thereof. For instance, the system 300 (e.g., wearable device 104, glucose monitoring device 305, user device 106, servers 110) may cause an insulin pump to output a specified volume/amount of insulin based on the blood glucose data 302, based on impacts of the blood glucose data 302 on the physiological data 301, based on impacts of the physiological data 301 on the blood glucose data 302, or any combination thereof. In some aspects, the system 300 may determine the specific volume/amount of insulin to be delivered based on data/analysis collected or performed by the system 300 (e.g., based on the blood glucose data 302).

In some aspects, the system 300 may perform the various analyses, calculations, and functions described in real-time or near-real time in order to identify potentially dangerous medical conditions (e.g., high/low blood glucose levels), and to take actions to better monitor for (and alert the user of) such medical conditions. For example, the system 300 may identify a high or low blood glucose level (and/or physiological characteristics that indicate that the user's blood glucose level is likely to become too high or too low), and may adjust operational parameters of the wearable device 104 and/or glucose monitoring device 305 and/or parameters of an insulin pump (while the high/low blood glucose condition is persisting). That is, the system 300 may be configured to identify whether a user falls into a particular class, category, or group (e.g., high blood glucose category, low blood glucose category), and may provide specific, actionable guidance or instructions based on the identified class, category, or group (e.g., prescribing a specific amount of insulin or carbohydrates, activating an insulin pump, etc.).

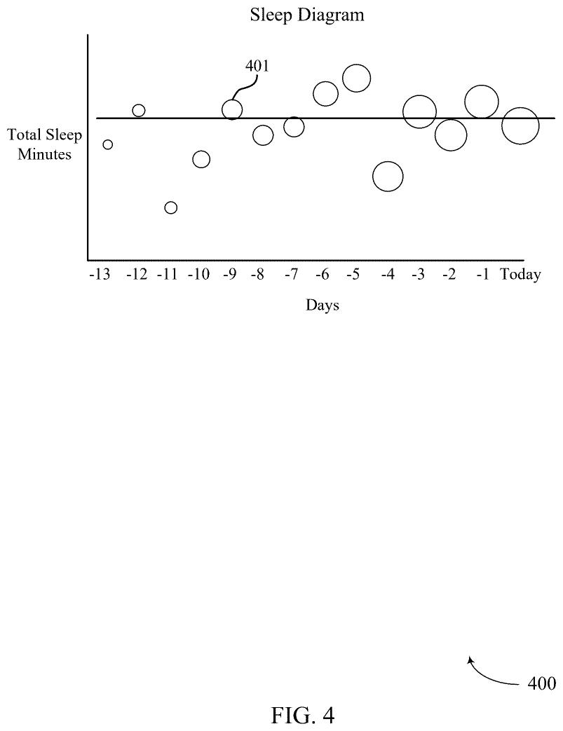

FIG. 4 shows an example of a sleep diagram 400 that supports techniques for evaluating impacts between physiological data and metabolic health in accordance with aspects of the present disclosure. Aspects of the sleep diagram 400 may implement, or be implemented by, aspects of the system 100, the system 200, the system 300, or any combination thereof.

It has been found that accumulation of sleep debt over a time period, such as a two week time period, may significantly impact a user's metabolic health. Sleep debt may lead to decreased insulin sensitivity, which can cause elevated average glucose, and higher glucose variability. Over time, this may contribute to higher risk of Type-2 diabetes.

The sleep diagram 400 in FIG. 4 illustrates a user's total sleep (in minutes) over a period of two weeks. The horizontal line across the sleep diagram 400 illustrates a weighted average (e.g., weighted average of sleep minutes per day), where the size of each data entry 401 indicates the relative weight given to the quantity/number of minutes slept on the corresponding night, where more recent nights are associated with larger weights, and nights further in the past are associated with smaller weights (e.g., weighted averages biased toward most recent night's sleep, as shown with the size of the data entries 401 increasing from left to right). For example, if a user sleeps poorly one night, their Sleep Score may be low the next day, but as time goes by, that outlier affects the average less and less given sleep (in minutes) of a particular day.

The sleep diagram 400 may be created or otherwise determined based on the physiological data 301 collected via the wearable device 104 of the system 300. In some cases, the sleep diagram 400 may be used to evaluate a user's sleep debt over time, where the system 300 may be able to determine relationships (e.g., effects, impacts) of the user's sleep debt on their blood glucose data 302. In some cases, days nearer to the current day may affect the weighted mean more than days further in the past. In other words, when evaluating the impact of the user's sleep debt on their blood glucose levels, the system 300 may be configured to “weight” physiological data that was collected more recently (e.g., in the last 48 hours) more heavily as compared to physiological data that was collected further in the past.

For example, the user's sleep debt may be used as a negative component in a calculation of composite scores. The higher the sleep debt, the more of a negative impact it has on e.g. Readiness Score.

The sleep debt may be inputted into a machine learning model where the machine learning model is trained to calculate one or more physiological parameters of the user based on weighting the physiological parameters in accordance with one or more predictive weights. The predictive weights may be based on the sleep debt. In other words, the user's physiological parameters may be weighted more or less based on the sleep debt, as the sleep debt may be a relative measure of how “trustworthy” the user's physiological data is for evaluating other physiological parameters. In such cases, the sleep debt may be used as an input into other models such as metabolic health, meal timing, and the like. For instance, high sleep debt may be indicative of stress or overtraining, and may therefore be used for calculating a user's cumulative stress over time, and therefore metabolic health. As described herein, a user may alter one or more behaviors in accordance with their metabolic health, such as to reduce the risk of burn-out, chronic illness, etc., which may help improve their overall health based on the current mental, physical, and emotional state of the user.

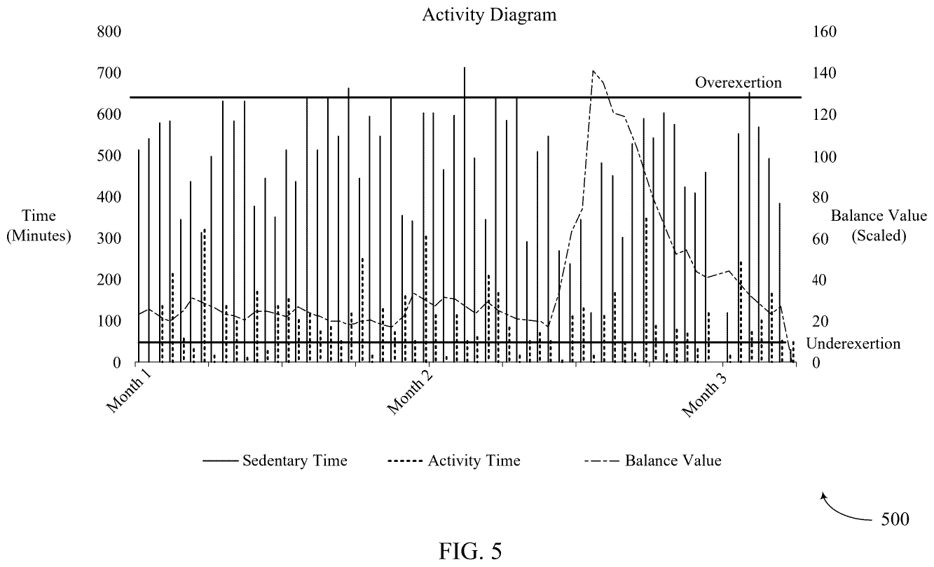

FIG. 5 shows an example of an activity diagram 500 that supports techniques for evaluating impacts between physiological data and metabolic health in accordance with aspects of the present disclosure. Aspects of the activity diagram 500 may implement, or be implemented by, aspects of the system 100, the system 200, the system 300, the sleep diagram 400, or any combination thereof.

Engaging in low to high-intensity activities, such as walking, cycling, or swimming, may enhance digestion and overall metabolic function. That is, “low to high” activity intensity may have an impact on the user's metabolic health. In this regard, the system 300 may be configured to determine a user's activity patterns/levels (e.g., based on the physiological data 301), and evaluate how the user's activity patterns/levels affect their blood glucose data 302 and overall health.

Additionally, excessive sedentary time may be linked to poorer metabolic health outcomes. Breaking up long periods of inactivity with short activity bursts can mitigate these effects. As such, the system 300 may be configured to provide recommendations as to how the user can adjust their activity patterns to improve their metabolic health. Further, while physical activity is beneficial to the user's overall health, excessively high intensity or prolonged exercise without adequate caloric intake can strain the metabolic system. In other words, balance is key. As such, by evaluating the user's activity patterns (and the impact of such activity patterns on the user's metabolic health), the system 300 may be configured to provide alerts or other recommendations to the user as to how they should adjust their activity patterns in light of their metabolic health.

For example, the activity diagram 500 illustrates the user's sedentary time and activity time over a period of two months, where the total sedentary time and activity time (in minutes) for each day are illustrated on the Y-axis on the left. The values of the sedentary time and activity time illustrated in the activity diagram 500 may be monitored and tracked via the system 300 (e.g., based on the physiological data 301). As described above, too much or too little activity (and/or too much or too little sedentary time) may strain the user's metabolic system. Therefore, in some aspects, the system 300 may also track a “balance value” that looks at the overall balance of the activity time and sedentary time, which is illustrated via the activity diagram 500. Specifically, a value of the “balance value” (e.g., sedentary-activity balance value) is illustrated on the Y-axis on the right side of the activity diagram 500. In some aspects, the balance value may take into account the total activity time and total sedentary time of the user over some time period (e.g., previous two months). Additionally, or alternatively, the balance value may take into account the intensity of the activity.

In effect, the balance value may track the user's overall balance between activity and relaxation/rest to determine whether the user is in a state of overexertion or underexertion. In some aspects, the values of the overexertion threshold and the underexertion thresholds illustrated in the activity diagram 500 may be tailored to each user, and may be based on previous physiological data 301 collected from the user.

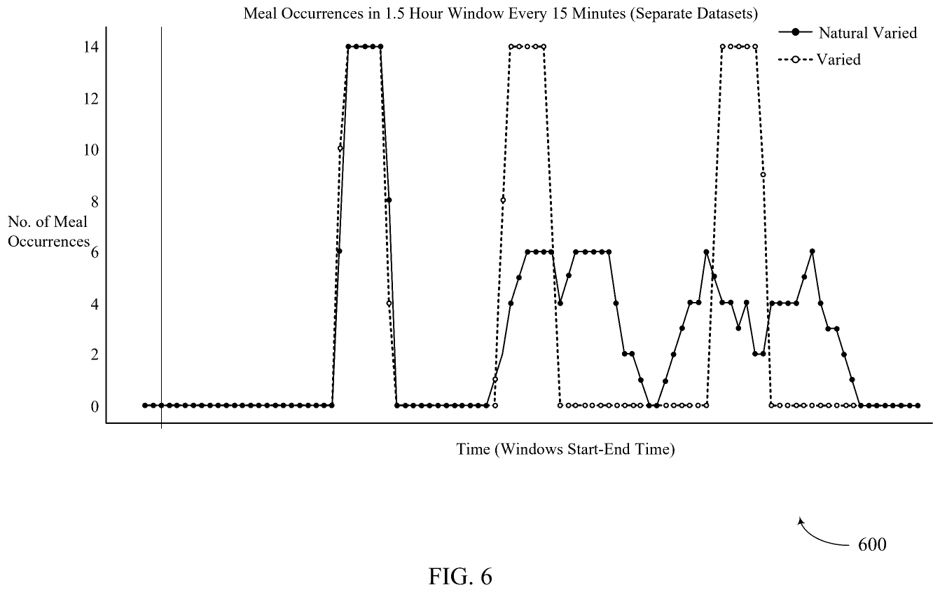

FIG. 6 shows an example of a meal timing diagram 600 that supports techniques for evaluating impacts between physiological data and metabolic health in accordance with aspects of the present disclosure. Aspects of the meal timing diagram 600 may implement, or be implemented by, aspects of the system 100, the system 200, the system 300, the sleep diagram 400, the activity diagram 500, or any combination thereof.

The meal timing diagram 600 illustrates a first curve labeled “varied,” which illustrates an “ideal situation” where a user eats within very specific time windows, and a second curve labeled “natural varied,” which illustrates a “real world” example of how most users eat in real life (e.g., more variation with meal timing.). As such, the difference between the respective curves illustrated in the meal timing diagram 600 refers to the time variation between main or primary meals. Maintaining a balance between the energy a user consumes through food and the energy the user expends through physical activities is crucial for metabolic health. As such, in some cases, the system 300 may be configured to track or monitor this balance between food consumption and exercise over time to further evaluate the relationship between the user's physiological data 301 and blood glucose data 302, as shown via the meal timing diagram 600. By monitoring this balance over time (rather than looking at individual daily values), techniques described herein may offer a more holistic view on the user's body functions.

In some cases, the system 300 may be configured to monitor the user's eating and drinking habits, and provide insights as to how such eating/drinking habits affect the user's metabolic health (e.g., how such eating/drinking habits affect the user's blood glucose data 302). For example, the system 300 may be configured to identify eating/drinking gestures (e.g., via motion data within the physiological data 301) in order to identify when the user eats a meal, or otherwise consumes food or drink. Consuming more energy than expended leads to storage of the excess as fat, increasing the risk of weight gain, obesity, and associated metabolic disorders. Additionally, regularly consuming less energy than your body expends can lead to energy deficits, which may affect muscle mass, slow down metabolism, and impair bodily functions and recovery processes.

It has been found that the concept of “food quality” is broader than the glucose response caused by the food we consume. As with everything, a single “bad” meal here and there is relatively harmless. Rather, preventing such bad meals from becoming a frequent habit is important for maintaining our overall health. Ideally, users should prioritize unsaturated fats (e.g., nuts, seeds, fish), and limit trans and saturated fats (e.g., processed foods, high-fat dairy, fatty meat). This may help maintain healthy blood lipids and reduce the risk of cardiometabolic disease. Users should also consume fiber-rich foods (e.g., vegetables, whole grains) to slow sugar absorption, enhance satiety, lower cholesterol, and support a healthy digestive system. Further, users should aim to minimize added sugars to prevent blood sugar spikes, insulin resistance, and associated risks of obesity, Type 2 diabetes, and cardiovascular disease. As such, users should focus on nutrient-dense, low-sugar foods for metabolic health.

It has also been found that the timing and/or consistency of food/drink consumption can have a large impact on the user's overall metabolic health. In particular, the timing of a user's meal consumption relative to their chronotype may affect how well they digest and process their food. As such, in some aspects, the system 300 may be configured to determine the user's chronotype (e.g., based on the physiological data 301), and may evaluate how the user's chronotype affects their metabolic health/blood glucose levels. Further, the system 300 may provide guidance/recommendations for adjusting the user's eating/drinking schedule to align with their chronotype to guide the user towards a healthier meal rhythm.

At least two of a user's main meals should happen in the same time window. Regular meal times can help regulate the body's digestive processes and help a user's digestive system get into a predictable rhythm. This helps with satiety control and avoiding cravings. Additionally, eating too close to the bedtime may have a negative impact on the user's blood glucose levels and metabolic health (chronotype specific). In particular, digestion slows down during the night, so late heavy meals should be avoided. In this regard, the system 300 may be configured to use the user's chronotype to determine the optimal eating windows, and to provide alerts when the system 300 detects that the user consumed a meal that negatively affected their blood glucose levels.

Taken together, and referring to the meal timing diagram 600, the system 300 may track when the user consumes meals throughout the day, such as based on tags inputted by the user in the wearable application 250, based on eating or drinking gestures identified within the physiological data 301 (e.g., identifying a “drinking” gesture by identifying that the user raised their hand up to their mouth), based on the blood glucose data 302, or any combination thereof. In this regard, the system 300 may be configured to “learn” how the timing of the user's food and drink consumption affects their physiological data 301, their blood glucose data 302, or both. For instance, by tracking the timing of the user's meal consumption, the system 300 may be configured to identify that eating too close to the user's bedtime results in a detrimental effect to the user's blood glucose data 302.

In some aspects, the system may be configured to provide guidance and alerts regarding the relative timing of meals to help the user optimize the timing of their food and drink consumption. In some cases, the system 300 may trigger alerts or guidance at specific times based on identified effects of meal timing, and/or based on trigger conditions that may increase the effectiveness of such guidance. For example, the system 300 may identify that the user is in the kitchen within an hour of their bedtime (such as based on signal strength measurements performed by the wearable device 104), and may trigger alerts to remind the user that eating too close to bedtime may detrimentally affect their overall health. Triggering such alerts at the specific time (e.g., shortly before bed) and in the specific location (e.g., while the user is in the kitchen) may increase the likelihood that the user will follow the guidance/alert, thereby improving their overall health.

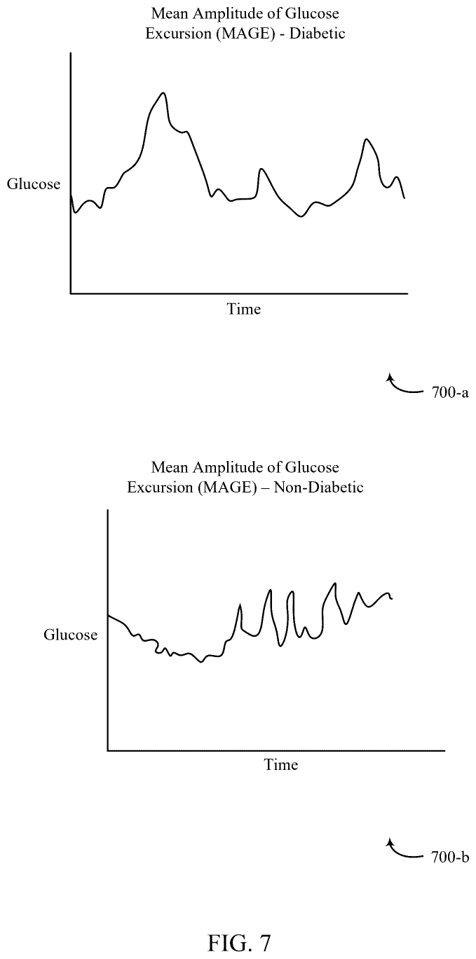

FIG. 7 shows examples of glucose diagrams 700-a, 700-b that support techniques for evaluating impacts between physiological data and metabolic health in accordance with aspects of the present disclosure. Aspects of the glucose diagrams 700-a, 700-b may implement, or be implemented by, aspects of the system 100, the system 200, the system 300, the sleep diagram 400, the activity diagram 500, the meal timing diagram 600, or any combination thereof.

In some cases, a user may consume the same meal at the same time on different days, where that same meal may have a different metabolic/glucose response on the respective days. That is, eating the same meal at the same time may lead to a highly different glucose response on different days. As such, using traditional glucose monitoring devices, users may be required to guess or make other assumptions as to why their blood glucose levels vary the way they do. Comparatively, by evaluating the user's physiological data 301 and blood glucose data 302 in a coordinated manner, (for example, determining connections between the variables, and determining the effect of the connections on further variables), aspects of the present disclosure may be used to provide scientifically valid metrics to understand the big picture. The approach has the effect of a more accurate, efficient, reliable, and faster informed evaluation of metabolic health.

To this end, some variability of the user's blood glucose levels is expected. However, over a longer time span, the average variability of glucose should remain fairly constant. As such, in some cases, the system 300 may be configured to evaluate various variability metrics of the blood glucose data 302 over time, such as the standard deviation or the mean amplitude of glucose excursion (MAGE) of the blood glucose data 302, as shown in the glucose diagrams 700-a, 700-b. In some cases, long-term changes (e.g., over weeks or months or years) of such variability metrics of the blood glucose data 302 may be a sign of the user developing diabetes, gestational diabetes or pre-diabetes. As such, the system 300 may be configured to evaluate such metrics over time and provide the user with alerts or other information to take actionable steps to improve their metabolic health.

For example, monitoring both blood glucose data 302 and other biometric data (e.g., physiological data 301) during pregnancy may enable early detection and management of gestational diabetes. Continuous glucose monitoring (CGM) may help identify abnormal glucose patterns in the blood glucose data 302 that may indicate impaired glucose tolerance. Evaluating the blood glucose data 302 and other biometric data (e.g., weight gain, blood pressure, heart rate variability, physical activity, sleep quality, and the like) may provide a more comprehensive picture of maternal health.

By integrating the blood glucose data 302 and other biometric data into the system 300, healthcare providers or digital monitoring systems may detect trends and flag early warning signs, such as elevated fasting glucose levels, spikes after meals, or excessive weight gain, thereby enabling timely interventions, such as dietary changes, physical activity adjustments, or further diagnostic testing. Early detection of gestational diabetes through a multifactorial monitoring approach helps reduce risks for both mother and baby, including preeclampsia, preterm birth, and future metabolic disorders.

Additionally, non-nutritional factors may be important contributors to the variability in the user's blood glucose levels. Daily fluctuations in glucose may be affected by sleep, activity, stress, and more. By keeping an eye on long-term averages, the system 300 may be configured to find comprehensive lifestyle patterns that support stable glucose control. That is, the system 300 may be configured to monitor the user's sleeping patterns, activity patterns, stress levels, and more, and identify how such physiological characteristics may be affecting (or are expected to affect) the user's blood glucose levels.

The system may identify a range of the user's blood glucose levels. For example, the system may determine whether the user's blood glucose data is within an optimal range, a good range, a fair range, or an elevated range. The system may determine a quantity of minutes that the user's blood glucose levels are within each range, a start time and an end time that the user's blood glucose levels are within each range, or both. In some cases, the system may provide suggestions/instructions on how to manage the blood glucose levels and minimize a time above range (e.g., within the elevated range). In other examples, the system may provide suggestions on how to maintain the blood glucose levels within the optimal range for example, given the range.

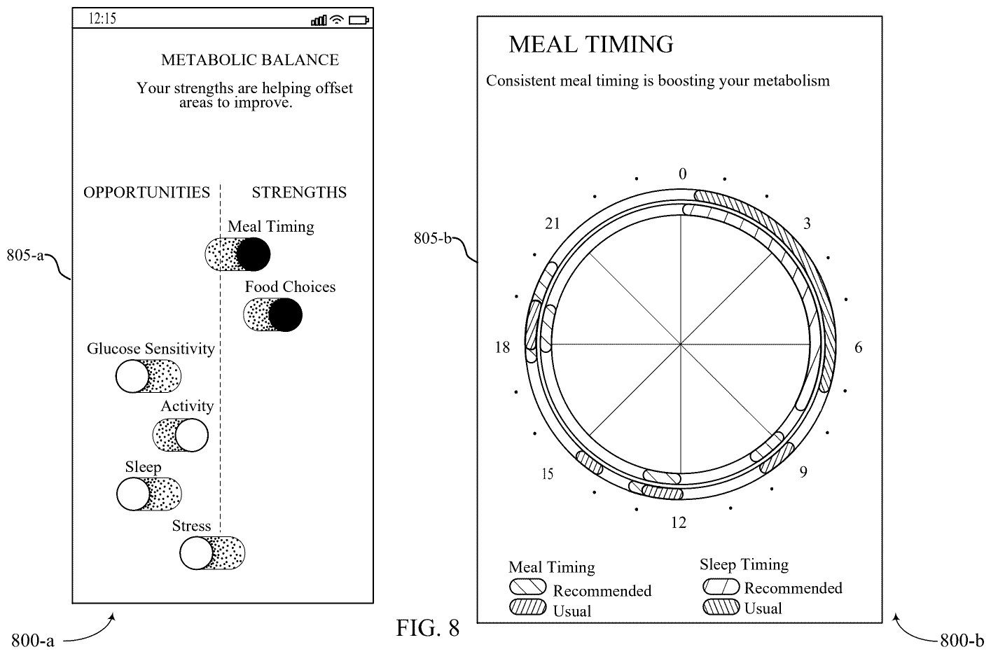

In some cases, the system may recommend a medication to lower or increase blood glucose levels based on the time above range. In such cases, the system may calculate a dosage of medication (e.g., insulin, sulfonylureas, biguanides, and the like) to lower the blood glucose levels of the user based on determining that the user's blood glucose data is above range. The system may instruct, via the user device associated with the wearable device, the user to intake the dosage of medication based on a quantity of minutes that the user's blood glucose data is above range. For example, the user device may indicate via a message, “It looks like your blood glucose levels have been elevated the past two hours after your meal. Take one unit of insulin to lower your blood sugar levels and check back again in one hour,” as further described with reference to FIG. 8. Additionally, or alternatively, as described previously herein, the system 300 may be configured to trigger an insulin pump or other medical device to administer a specific amount of insulin.

In such cases, the system may instruct the user to administer a treatment to minimize the time above range and lower the blood glucose levels from the elevated range to the optimal range, fair range, or good range. In other examples, the system may instruct the user to administer a treatment to minimize the time below range and increase the blood glucose levels to the optimal range, fair range, or good range. In such cases, the system may calculate a dosage of carbohydrates to increase the blood glucose levels of the user based on determining that the user's blood glucose data is below range. The system may instruct, via the user device associated with the wearable device, the user to intake the carbohydrates based on the blood glucose data being below range. For example, the user device may indicate via a message, “It looks like your blood glucose levels are low. Take fifteen grams of fast-acting carbohydrates to increase your blood sugar levels. Glucose tablets, juice, or soda are all great examples of fast-acting carbohydrates.” Example messaging may be further described with reference to FIG. 8.

In other examples, the system may transmit a signal to a device coupled with the user (e.g., an insulin pump) to administer a calculated volume/amount of insulin to the user and lower the blood glucose levels. In other examples, the system may transmit a signal to the device coupled with the user (e.g., the insulin pump) to stop administering insulin to the user to increase the blood glucose levels. In such cases, the signal transmitted to the user device may cause the device to override instructions to administer insulin and rather stop or delay administering insulin to the user.

FIG. 8 shows an example of GUIS 800 (e.g., a GUI 800-a and a GUI 800-b) that support techniques for evaluating impacts between physiological data and metabolic health in accordance with aspects of the present disclosure.

As described previously, with reference to FIG. 3, the system 300 may provide one or more insights to a user based on their metabolic health. For example, the system 200 may determine an overall (e.g., baseline) metabolic health for the user (e.g., Metabolic Health Score). In some cases, the overall metabolic health may be determined for the user based on physiological data, lifestyle data, or both collected (e.g., acquired, measured) from the user during a first time interval e.g., 1 week, 2 weeks, 1 month, etc.). That is, the system 300 may collect first physiological data, first lifestyle data, or both during the first time interval to determine one or more first physiological characteristics, one or more first lifestyle characteristics, or both, respectively, and may determine the overall metabolic health for the user based on the one or more first physiological characteristics, one or more first lifestyle characteristics, or both. In such cases, the one or more first physiological characteristics may be associated with sleep of the user (e.g., a sleep quality of the user), stress of the user (e.g., a stress level of the user), activity of the user (e.g., an activity level of the user), a glucose sensitivity of the user, recovery of the user (e.g., recovery time), or any combination thereof. The one or more first lifestyle characteristics of the user may be associated with one or more meals consumed by the user (e.g., food choices, nutritional value), a meal timing associated with each of the one or more meals, activity timing of the user, one or more activity patterns of the user, sleep timing of the user, or any combination thereof.

Additionally, the system 300 may acquire (e.g., collect via a user device, a wearable device, or both) second physiological data, second lifestyle data, or both, from the user and may determine one or more second physiological characteristics, one or more second lifestyle characteristics, or both, respectively, based on the second physiological data, the second lifestyle data, or both. In some examples, the system 300 may acquire the second physiological data, the second lifestyle data, or both, during a second time interval (e.g., equal to or different from the first time interval) after the second time interval. For example, the system 300 may collect the second physiological data, the second lifestyle data, or both, during 1 week that occurs 1 month after the first time interval. In other words, the system 300 may evaluate (e.g., determine) the metabolic health of the user (e.g., impacts to metabolic health, changes in metabolic health) monthly (e.g., for one week each month). In some other cases, the second time interval may be continuous, such that the system 300 may continuously (e.g., each day each week) evaluate the metabolic health of the user.

Additionally, the system 300 may determine one or more impacts on the overall metabolic health of the user based on the one or more second physiological characteristics, the one or more second lifestyle characteristics, or both. That is, the system 300 may determine a change (and details thereof) in overall metabolic health (e.g., a change in Metabolic Health Score), may determine a new overall metabolic health of the user (e.g., an updated Metabolic Health Score), or both, based on the one or more second physiological characteristics, the one or more second lifestyle characteristics, or both. For example, the system 300 may determine that an average meal timing of the user (e.g., during the second time interval) resulted in a positive change to the overall metabolic health of the user while an average stress level of the user (e.g., during the second time interval) resulted in a negative change to the overall metabolic health of the user.

Additionally, or alternatively, the system 300 may generate and display (e.g., via the user device) one or more recommendations for the user based on the one or more impacts on the overall metabolic health of the user. That is, the system 300 may identify one or more recommended physiological characteristics, one or more recommended lifestyle characteristics, or both, (e.g., the physiological data, the lifestyle data, or both) that may result in a positive impact to the overall metabolic health of the user and may display the one or more recommended physiological characteristics, the one or more recommended lifestyle characteristics, or both. For example, as depicted in the GUI 800-b, the system may identify that shifting meal timing of the user and sleep timing of the user may result in positive impacts to the overall metabolic health (e.g., metabolism) of the user, such that the system 300 may display an indication of the recommended shift in sleep timing and the recommended shift in meal timing to the user. Additionally, or alternatively, as depicted in the GUI 800-b may display an indication of the user's actual (e.g., usual) sleep timing and actual meal timing compared to the recommended sleep timing and recommended meal timing, respectively.

In some examples, the GUI 800 illustrates a series of application pages 805 (e.g., application page 805-a and application page 805-b) which may be displayed to a user via the GUI 800. The server of the system may cause the GUI 800 of the user device (e.g., mobile device) to display inquiries of whether the user activates a metabolic health tracking mode and wants to track their mealtimes, glucose, and the like (e.g., via application page 805). In such cases, the system may generate a personalized tracking experience on the GUI 800 of the user device to evaluate metabolic health.

Continuing with the examples above, prior to identifying one or more impacts that the physiological characteristics had on blood glucose data, the user may be presented with an application page upon opening the wearable application. The application page 805 may display a request to activate the metabolic health tracking mode and enable the system to track the metabolic health of the user (e.g., including tracking blood glucose data, meal, and the like). In such cases, the application page 805 may display an invitation card where the users are invited to enroll in the metabolic health tracking applications and tracking blood glucose data and/or meals. The application page 805 may display a prompt to the user to verify whether the blood glucose data and/or meals may be tracked or dismiss the message if the blood glucose data and/or meals is not tracked. The system may receive an indication of whether the user selects to opt-in to tracking the metabolic health of the user or opt-out to tracking the metabolic health of the user.

The user may be presented with an application page 805 upon selecting “yes” to tracking the metabolic health. The application page 805 may display a prompt to the user to connect the application with the glucose monitoring device. In some case, the application page 805 may display a series of steps to connect the glucose monitoring device with the user device in order for the user device to track the blood glucose data and identify one or more impacts that the physiological characteristics had on the blood glucose data. For example, the system may receive, via the user device, a confirmation that the glucose monitoring device is communicatively coupled with the user device. In some cases, the application page 805 may display a prompt to order the glucose monitoring device.

In some cases, the application page 805 may display a glucose diagram, as described with reference to FIG. 7. In some examples, the application page 805 may overlay one or more physiological characteristics on top of the glucose diagram. For example, the application page 805 may present the blood glucose data and a stress level of the user, an activity level of the user, a recovery of the user, a meal of the user, sleep data associated with the user, or a combination thereof.

In some cases, the application page 805 may display a prompt to the user to input one or more tags associated with the blood glucose data. For example, the user may be prompted to input a time of day that the user eats and/or drinks, a description of what the user eats and/or drinks (e.g., including a type of food and/or a drink, a quantity, a duration of time that the user was eating and/or drinking, and the like). Based on the input from the user, the system may overlay the tags on the glucose diagram. In some cases, the system may deliver personalized messaging based on the blood glucose data. For example, the system may message “Discover the effect of your actions. Scroll through the graph to discover events that affect your glucose levels.” Taggable events received via a wearable device, user device, or both, may enable better prediction of impacts.

Based on the blood glucose data, the system may alert a healthcare provider, provide the user with treatment suggestions and/or referrals to a healthcare provider, provide the user with tools to adjust or maintain the blood glucose data (e.g., take a quick walk, watch this video, read this article, recommended meal plan). For example, the system may provide suggestions on how to manage the blood glucose data and minimize a time above range. As users complete some of their suggested activities they may receive feedback from the system by seeing in real time how their actions mitigate their blood glucose data and/or change their blood glucose data, the content delivered, the messaging, or a combination thereof. In such cases, users may take control of their health by understanding what contributes and what does not contribute to their blood glucose data.

In some cases, the system may deliver personalized messaging based on the time above range. For example, the system may message “Your glucose levels have been optimal today. Focus on balanced meals and activity to help maintain your glucose levels in the optimal range.” The time above range card may indicate a quantity of minutes that the user's blood glucose data is within each range. For example, the system may determine a quantity of minutes that the user's blood glucose data is within an optimal range, a good range, a fair range, or an elevated range. In some cases, the tags may indicate a quantity of minutes that the blood glucose data was above range based on what and when the user eats or drinks. In some cases, the message may indicate “Research shows that as little as 2 minutes of walking after a meal can help mitigate glucose spikes, but a 10-20 minute walk is ideal.”

The server of the system may generate a message for display on the GUI 800 on a user device that indicates the one or more impacts that the one or more physiological characteristics had on the blood glucose data. For example, the server of the system may cause the GUI 800 of the user device (e.g., mobile device) to display a message associated with one or more impacts that the one or more physiological characteristics had on the blood glucose data (e.g., via application page 805). In such cases, the system may output the one or more impacts that the one or more physiological characteristics had on the blood glucose data on the GUI 800 of the user device.

Additionally, in some implementations, the application page 805 may display one or more scores (e.g., Sleep Score, Readiness Score, Activity Score, etc.) for the user for the respective day. Moreover, in some cases, the one or more impacts that the one or more physiological characteristics had on the blood glucose data may be used to update (e.g., modify) one or more scores associated with the user (e.g., Sleep Score, Readiness Score, etc.). That is, data associated with the one or more impacts that the one or more physiological characteristics had on the blood glucose data may be used to update the scores for the user for the following calendar days. In such cases, the system may notify the user of the score update via an alert.

In some implementations, the system may notify, via an alert or message, a user if the blood glucose data is in the optimal range, the good range, the fair range, or the elevated range. For example, the system may detect the blood glucose data is in the elevated range and recommend related action/and or tags. In some implementations, the system may combine information from tags and user data. In response to detection of the blood glucose being in the elevated range, a device may alert users when they may want to exercise, pay close attention to specific symptoms, take a prescribed medication, or consult their physician. The combination of multiple, continuous, high quality signals with tagged symptoms in the application may provide the basis for an interactive algorithm that takes into account deviations from one's prior history as well as deviations from a typical profile. Tags surfaced to the user through the application may bring awareness to specific symptoms that the user might otherwise mistake for tiredness, fatigue, etc. Additional notifications may point to signals that require further attention, such as “You have been experiencing long periods of elevated blood glucose for a while now following your meals, have you discussed this with your physician?” or “You are experiencing long durations of elevated blood glucose levels, you may want to go on a walk, eat smaller portions, and incorporate active times in your schedule.”

For example, the system may provide, via glucose diagram, the user with a trend graph for the user's blood glucose data, other physiologic data, and/or mealtime data against a comparison graph of the blood glucose range considered “normal,” so that the user can understand their body and make informed choices about seeking medical care, altering lifestyle choice (e.g., diet, exercise, sleep, stress), and the like. In another example, the system may alert users when they should consider discussing their blood glucose data with a physician. In some implementations, the system may output a predicted risk score for conditions of diabetes.

Application page 805 may also include messages that include insights, recommendations, and the like associated with the blood glucose data. The server of the system may cause the GUI 800 of the user device to display a message and/or recommendation associated with the blood glucose data. As noted previously herein, an accurately predicted and/or identified impact of physical characteristics on the blood glucose data may be beneficial to a user's overall health and recovery process.

In some implementations, the system may provide additional insight regarding the user's predicted and/or identified one or more conditions of mental or emotional distress. For example, the application pages 805 may indicate one or more physiological parameters (e.g., contributing factors) which resulted in the user's blood glucose data. In other words, the system may be configured to provide some information or other insights regarding the contributing factors to the blood glucose data. Personalized insights may indicate aspects of collected physiological data (e.g., contributing factors within the physiological data) which were used to generate the impact of physical characteristics on the blood glucose data.

Continuing with the examples above, the user may be presented with an application page 805 upon selecting “yes” to tracking the metabolic health and/or meals. In some cases, the application page 805 may display a prompt to the user to input one or more goals associated with meal logging. For example, the user may be prompted to input a main goal for logging meals such as “Make better food choices, improve my meal timing, increase protein intake, balance blood sugar, manage cravings, gain muscle, and/or lose weight.” In some cases, the application page 805 may display a prompt to the user to input one or more dietary restrictions. For example, the user may be prompted to input a dietary restriction associated with meals such as “I eat everything, pescatarian, vegetarian, vegan, paleo, ketogenic, and/or carnivore.”

In some cases, the application page 805-b may display a circadian rhythm timing diagram, as described with reference to FIG. 8. In some examples, the application page 805-b may overlay one or more physiological characteristics on top of the circadian rhythm timing diagram, including meals. For example, the application page 805-b may present recommended meal timing for the user, usual meal timing for the user, an indication of a meal, or a combination thereof. The circadian rhythm timing diagram may indicate a “wind down period” where it is not suggested to eat heavy meals during this time.

In some cases, the application page 805 may display a prompt to the user to input one or more tags associated with the meal. For example, the user may be prompted to input a time of day that the user eats and/or drinks, a description of what the user eats and/or drinks (e.g., including a type of food and/or a drink, a quantity, a duration of time that the user was eating and/or drinking, and the like). Based on the input from the user, the system may overlay the tags on the circadian rhythm timing diagram.

In some examples, the user may input a meal by taking a photograph of the meal using the user device. For example, the system may analyze the photograph (such as by inputting the image/photograph into a machine learning classifier/model) to determine a meal type (e.g., breakfast, lunch, dinner, dessert, snack, drink), a time of the meal, nutritional content of the meal, or a combination thereof. The nutritional content of the meal may be an example of types of foods and/or nutritional breakdown for each type of food. The nutritional breakdown may include at least protein, fiber, processing levels, added sugars, total fats, total carbohydrate, or a combination thereof. For each nutritional breakdown, the system may indicate whether the meal includes a low amount, a moderate amount, or a high amount. In some cases, the system may deliver messaging such as “Protein is a macronutrient essential for building and repairing tissues, muscles, and organs. For those engaging in physical activities or strength training, protein intake supports muscle repair and helps maintain lead body mass.” In some examples, the user may input a meal by logging a favorite meal and selecting the meal from a drop-down menu. The user may manually input a meal by logging the time, date, meal type, meal content, and the like.

In some cases, the system may deliver personalized messaging based on the meal data. For example, the system may message “Great choice for breakfast! The protein and fiber combo helps keep you energized and your glucose steady.” In other examples, the system may message “Logging your meals consistently helps you uncover the nutritional content of your meal and understand how your eating habits affect your circadian rhythm and your body's internal processes.”

The system may send personalized messaging based on the user's input for their main goals for logging meals. For example, the system may message “Regular eating habits can help maintain stable blood sugar and steady energy levels throughout the day. Eating at consistent times supports your body's internal clock, or circadian rhythm, making it easier to fall and stay asleep at night.”

Based on the meals data, the system may alert a healthcare provider, provide the user with meal timing suggestions and/or referrals to a healthcare provider, provide the user with tools to adjust or maintain the meal timing, meal content, and the like (e.g., watch this video, read this article, recommended meal plan). For example, the system may provide suggestions on how to manage the meal timing and minimize a time above range related to the user's blood glucose data.

The server of the system may generate a message for display on the GUI 800 on a user device that indicates the one or more impacts that the meal had on the blood glucose data, both based on the content of the meal and the timing of the meal. For example, the server of the system may cause the GUI 800 of the user device (e.g., mobile device) to display a message associated with one or more impacts that the meal had on the blood glucose data (e.g., via application page 805). In such cases, the system may output the one or more impacts that the meal had on the blood glucose data on the GUI 800 of the user device. In some cases, the system may deliver personalized messaging based on the time above range. For example, the system may message “After your breakfast, your time above your blood glucose range was 10 minutes, which is below your average time above your blood glucose range after breakfast (15-20 minutes).”

In some implementations, the system may notify, via an alert or message, a user if the meal content (e.g., nutritional breakdown) is in the low amount, moderate amount, and/or high amount. For example, the system may detect the total carb is in the high amount and recommend related action/and or tags. In some implementations, the system may combine information from tags and user data. In response to detection of the total carbs, for example, being in a high range/amount, a device may alert users when they may want to exercise, go to bed, recommended time to eat the meal in the future, or consult their dietician. Additional notifications may point to signals that require further attention, such as “You have been eating large, heavy meals right before bed and it seems to be affecting your sleep. You may want to try to eat smaller portions and/or eat earlier in the day.” As noted previously herein, an accurately predicted and/or identified impact of meals on the blood glucose data, the user's sleep, and the like may be beneficial to a user's overall health and recovery process.

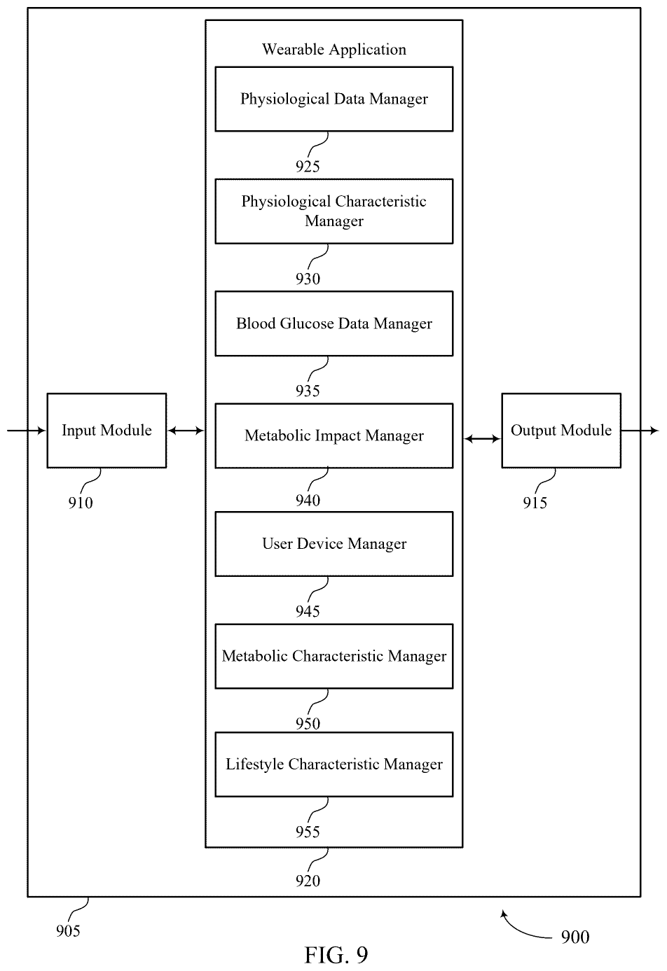

FIG. 9 shows a block diagram 900 of a device 905 that supports techniques for evaluating impacts between physiological data and metabolic health in accordance with aspects of the present disclosure. The device 905 may include an input module 910, an output module 915, and a wearable application 920. The device 905, or one or more components of the device 905 (e.g., the input module 910, the output module 915, the wearable application 920), may include at least one processor, which may be coupled with at least one memory, to support the described techniques. Each of these components may be in communication with one another (e.g., via one or more buses).

The input module 910 may provide a means for receiving information such as packets, user data, control information, or any combination thereof associated with various information channels (e.g., control channels, data channels, information channels related to illness detection techniques). Information may be passed on to other components of the device 905. The input module 910 may utilize a single antenna or a set of multiple antennas.

The output module 915 may provide a means for transmitting signals generated by other components of the device 905. For example, the output module 915 may transmit information such as packets, user data, control information, or any combination thereof associated with various information channels (e.g., control channels, data channels, information channels related to illness detection techniques). In some examples, the output module 915 may be co-located with the input module 910 in a transceiver module. The output module 915 may utilize a single antenna or a set of multiple antennas.

For example, the wearable application 920 may include a physiological data manager 925, a physiological characteristic manager 9930, a blood glucose data manager 935, a metabolic impact manager 940, a user device manager 945, a metabolic characteristic manager 950, or any combination thereof. In some examples, the wearable application 920, or various components thereof, may be configured to perform various operations (e.g., receiving, monitoring, transmitting) using or otherwise in cooperation with the input module 910, the output module 915, or both. For example, the wearable application 920 may receive information from the input module 910, send information to the output module 915, or be integrated in combination with the input module 910, the output module 915, or both to receive information, transmit information, or perform various other operations as described herein.

The wearable application 920 may support evaluating metabolic health in accordance with examples as disclosed herein. The physiological data manager 925 may be configured as or otherwise support a means for acquiring, from a wearable device using one or more processors, physiological data collected from a user via the wearable device, the physiological data comprising at least PPG data. The physiological characteristic manager 930 may be configured as or otherwise support a means for determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof. The blood glucose data manager 935 may be configured as or otherwise support a means for acquiring, from a glucose monitoring device using the one or more processors, blood glucose data collected from the user via the glucose monitoring device. The metabolic impact manager 940 may be configured as or otherwise support a means for identifying, using the one or more processors, one or more impacts that the one or more physiological characteristics had on the blood glucose data. The user device manager 945 may be configured as or otherwise support a means for transmitting a signal to a user device to cause the user device to convey information to the user associated with the one or more impacts. The user device manager 945 may be configured as or otherwise support a means for transmitting a signal to a user device based on the one or more impacts to cause the user device take action responsive to/based on the one or more impacts.

Additionally, or alternatively, the wearable application 920 may support evaluating metabolic health in accordance with examples as disclosed herein. The blood glucose data manager 935 may be configured as or otherwise support a means for acquiring, from a glucose monitoring device using one or more processors, blood glucose data collected from a user via the glucose monitoring device. The metabolic characteristic manager 950 may be configured as or otherwise support a means for determining, using the one or more processors, one or more metabolic characteristics of the user based at least in part on the blood glucose data. The physiological data manager 925 may be configured as or otherwise support a means for acquiring, from a wearable device using the one or more processors, physiological data collected from the user via the wearable device, the physiological data comprising at least PPG data. The physiological characteristic manager 930 may be configured as or otherwise support a means for determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof. The metabolic impact manager 940 may be configured as or otherwise support a means for identifying, using the one or more processors, one or more impacts that the one or more metabolic characteristics had on the one or more physiological characteristics. The user device manager 945 may be configured as or otherwise support a means for transmitting a signal to a user device to cause the user device to convey information to the user associated with the one or more impacts. The user device manager 945 may be configured as or otherwise support a means for transmitting a signal based on the one or more impacts to a user device to cause the user device to interact with the user based on the one or more impacts. The interaction may be an alert, instruction, or a physical interaction such as a vibrate or noise, for instance.

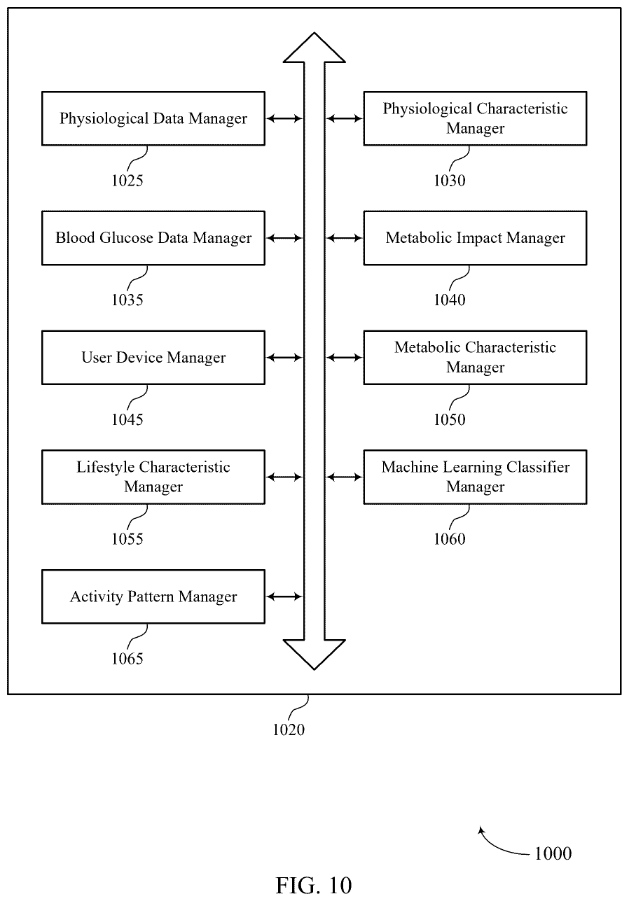

FIG. 10 shows a block diagram 1000 of a wearable application 1020 that supports techniques for evaluating impacts between physiological data and metabolic health in accordance with aspects of the present disclosure. The wearable application 1020 may be an example of aspects of a wearable application or a wearable application 920, or both, as described herein. The wearable application 1020, or various components thereof, may be an example of means for performing various aspects of techniques for evaluating impacts between physiological data and metabolic health as described herein. For example, the wearable application 1020 may include a physiological data manager 1025, a physiological characteristic manager 1030, a blood glucose data manager 1035, a metabolic impact manager 1040, a user device manager 1045, a metabolic characteristic manager 1050, a machine learning classifier manager 1055, an activity pattern manager 1060, or any combination thereof. Each of these components, or components of subcomponents thereof (e.g., one or more processors, one or more memories), may communicate, directly or indirectly, with one another (e.g., via one or more buses).

The wearable application 1020 may support evaluating metabolic health in accordance with examples as disclosed herein. The physiological data manager 1025 may be configured as or otherwise support a means for acquiring, from a wearable device using one or more processors, physiological data collected from a user via the wearable device, the physiological data comprising at least PPG data. The physiological characteristic manager 1030 may be configured as or otherwise support a means for determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof. The blood glucose data manager 1035 may be configured as or otherwise support a means for acquiring, from a glucose monitoring device using the one or more processors, blood glucose data collected from the user via the glucose monitoring device. The metabolic impact manager 1040 may be configured as or otherwise support a means for identifying, using the one or more processors, one or more impacts that the one or more physiological characteristics had on the blood glucose data. The user device manager 1045 may be configured as or otherwise support a means for transmitting a signal to a user device to cause the user device to convey information to the user associated with the one or more impacts.

In some examples, the machine learning classifier manager 1055 may be configured as or otherwise support a means for inputting the physiological data, the one or more physiological characteristics, and the blood glucose data into a machine learning classifier, wherein the machine learning classifier is configured to output an indication of the one or more impacts.

In some examples, the physiological data manager 1025 may be configured as or otherwise support a means for identifying an eating gesture, a drinking gesture, or both, based at least in part on the motion data, wherein identifying the one or more impacts is based at least in part on identifying the eating gesture, the drinking gesture, or both.

In some examples, the one or more physiological characteristics comprise a sleep debt of the user. In some examples, the one or more impacts comprise an increase in the blood glucose data of the user.

In some examples, the blood glucose data manager 1035 may be configured as or otherwise support a means for identifying one or more changes in the blood glucose data. In some examples, the activity pattern manager 1060 may be configured as or otherwise support a means for identifying an activity pattern of the user during a time interval preceding the one or more changes in the blood glucose data, wherein the one or more impacts are based at least in part on a temporal relationship between the activity pattern and the one or more changes.

In some examples, to support identifying the one or more impacts, the physiological characteristic manager 1030 may be configured as or otherwise support a means for identifying a sleep period that the user is asleep based at least in part on the physiological data. In some examples, to support identifying the one or more impacts, the physiological data manager 1025 may be configured as or otherwise support a means for identifying one or more meals consumed by the user based at least in part on the physiological data, the blood glucose data, or both, wherein the one or more impacts are based at least in part on a temporal relationship between the sleep period and the one or more meals.

In some examples, the glucose monitoring device comprises a component of the wearable device.

In some examples, the wearable device comprises a wearable ring device.

Additionally, or alternatively, the wearable application 1020 may support evaluating metabolic health in accordance with examples as disclosed herein. In some examples, the blood glucose data manager 1035 may be configured as or otherwise support a means for acquiring, from a glucose monitoring device using one or more processors, blood glucose data collected from a user via the glucose monitoring device. The metabolic characteristic manager 1050 may be configured as or otherwise support a means for determining, using the one or more processors, one or more metabolic characteristics of the user based at least in part on the blood glucose data. In some examples, the physiological data manager 1025 may be configured as or otherwise support a means for acquiring, from a wearable device using the one or more processors, physiological data collected from the user via the wearable device, the physiological data comprising at least PPG data. In some examples, the physiological characteristic manager 1030 may be configured as or otherwise support a means for determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof. In some examples, the metabolic impact manager 1040 may be configured as or otherwise support a means for identifying, using the one or more processors, one or more impacts that the one or more metabolic characteristics had on the one or more physiological characteristics. In some examples, the user device manager 1045 may be configured as or otherwise support a means for transmitting a signal to a user device to cause the user device to convey information to the user associated with the one or more impacts.

In some examples, the machine learning classifier manager 1055 may be configured as or otherwise support a means for inputting the physiological data, the blood glucose data, the one or more physiological characteristics, and the one or more metabolic characteristics into a machine learning classifier, wherein the machine learning classifier is configured to output an indication of the one or more impacts.

In some examples, the physiological data manager 1025 may be configured as or otherwise support a means for identifying an eating gesture, a drinking gesture, or both, based at least in part on the motion data, wherein identifying the one or more impacts is based at least in part on identifying the eating gesture, the drinking gesture, or both.

In some examples, the blood glucose data manager 1035 may be configured as or otherwise support a means for identifying one or more changes in the blood glucose data. In some examples, the activity pattern manager 1060 may be configured as or otherwise support a means for identifying an activity pattern of the user during a time interval preceding the one or more changes in the blood glucose data, wherein the one or more impacts are based at least in part on a temporal relationship between the activity pattern and the one or more changes.

In some examples, the physiological data manager 1025 may be configured as or otherwise support a means for identifying a sleep period that the user is asleep based at least in part on the physiological data. In some examples, the physiological data manager 1025 may be configured as or otherwise support a means for identifying one or more meals consumed by the user based at least in part on the physiological data, the blood glucose data, or both, wherein the one or more impacts are based at least in part on a temporal relationship between the sleep period and the one or more meals.

In some examples, the glucose monitoring device comprises a component of the wearable device.

In some examples, the wearable device comprises a wearable ring device.

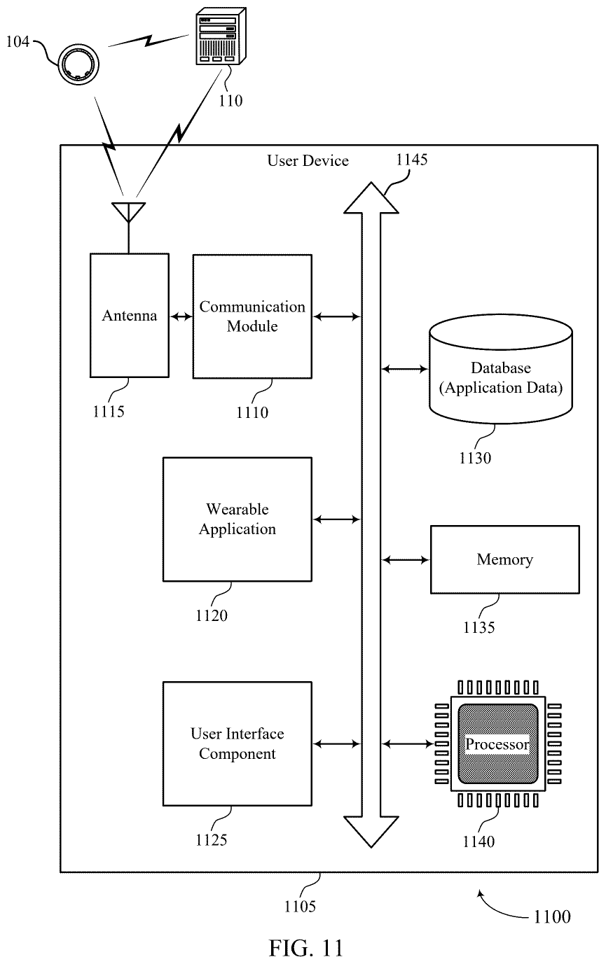

FIG. 11 shows a diagram of a system 1100 including a device 1105 that supports techniques for evaluating impacts between physiological data and metabolic health in accordance with aspects of the present disclosure. The device 1105 may be an example of or include components of a device 905 as described herein. The device 1105 may include an example of a user device 106, as described previously herein. The device 1105 may include components for bi-directional communications including components for transmitting and receiving communications with a wearable device 104 and a server 110, such as a wearable application 1120, a communication module 1110, one or more antennas 1115, a user interface component 1125, a database (application data) 1130, at least one memory 1135, and at least one processor 1140. These components may be in electronic communication or otherwise coupled (e.g., operatively, communicatively, functionally, electronically, electrically) via one or more buses (e.g., a bus 1145).

The communication module 1110 may manage input and output signals for the device 1105 via the antenna 1115. The communication module 1110 may include an example of the communication module 220-b of the user device 106 shown and described in FIG. 2. In this regard, the communication module 1110 may manage communications with the ring 104 and the server 110, as illustrated in FIG. 2. The communication module 1110 may also manage peripherals not integrated into the device 1105. In some cases, the communication module 1110 may represent a physical connection or port to an external peripheral. In some cases, the communication module 1110 may utilize an operating system such as iOS®, ANDROID®, MS-DOS®, MS-WINDOWS®, OS/2®, UNIX®, LINUX®, or another known operating system. In other cases, the communication module 1110 may represent or interact with a wearable device (e.g., ring 104), modem, a keyboard, a mouse, a touchscreen, or a similar device. In some cases, the communication module 1110 may be implemented as part of the processor 1140. In some examples, a user may interact with the device 1105 via the communication module 1110, user interface component 1125, or via hardware components controlled by the communication module 1110.

In some cases, the device 1105 may include a single antenna 1115. However, in some other cases, the device 1105 may have more than one antenna 1115, which may be capable of concurrently transmitting or receiving multiple wireless transmissions. The communication module 1110 may communicate bi-directionally, via the one or more antennas 1115, wired, or wireless links as described herein. For example, the communication module 1110 may represent a wireless transceiver and may communicate bi-directionally with another wireless transceiver. The communication module 1110 may also include a modem to modulate the packets, to provide the modulated packets to one or more antennas 1115 for transmission, and to demodulate packets received from the one or more antennas 1115.

The user interface component 1125 may manage data storage and processing in a database 1130. In some cases, a user may interact with the user interface component 1125. In other cases, the user interface component 1125 may operate automatically without user interaction. The database 1130 may be an example of a single database, a distributed database, multiple distributed databases, a data store, a data lake, or an emergency backup database.

The memory 1135 may include RAM and ROM. The memory 1135 may store computer-readable, computer-executable software including instructions that, when executed, cause the processor 1140 to perform various functions described herein. In some cases, the memory 1135 may contain, among other things, a BIOS which may control basic hardware or software operation such as the interaction with peripheral components or devices.

The processor 1140 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 1140 may be configured to operate a memory array using a memory controller. In other cases, a memory controller may be integrated into the processor 1140. The processor 1140 may be configured to execute computer-readable instructions stored in a memory 1135 to perform various functions (e.g., functions or tasks supporting a method and system for sleep staging algorithms).

The wearable application 1120 may support evaluating metabolic health in accordance with examples as disclosed herein. For example, the wearable application 1120 may be configured as or otherwise support a means for acquiring, from a wearable device using one or more processors, physiological data collected from a user via the wearable device, the physiological data comprising at least PPG data. The wearable application 1120 may be configured as or otherwise support a means for determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof. The wearable application 1120 may be configured as or otherwise support a means for acquiring, from a glucose monitoring device using the one or more processors, blood glucose data collected from the user via the glucose monitoring device. The wearable application 1120 may be configured as or otherwise support a means for identifying, using the one or more processors, one or more impacts that the one or more physiological characteristics had on the blood glucose data. The wearable application 1120 may be configured as or otherwise support a means for transmitting a signal to a user device to cause the user device to convey information to the user associated with the one or more impacts.

Additionally, or alternatively, the wearable application 1120 may support evaluating metabolic health in accordance with examples as disclosed herein. For example, the wearable application 1120 may be configured as or otherwise support a means for acquiring, from a glucose monitoring device using one or more processors, blood glucose data collected from a user via the glucose monitoring device. The wearable application 1120 may be configured as or otherwise support a means for determining, using the one or more processors, one or more metabolic characteristics of the user based at least in part on the blood glucose data. The wearable application 1120 may be configured as or otherwise support a means for acquiring, from a wearable device using the one or more processors, physiological data collected from the user via the wearable device, the physiological data comprising at least PPG data. The wearable application 1120 may be configured as or otherwise support a means for determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof. The wearable application 1120 may be configured as or otherwise support a means for identifying, using the one or more processors, one or more impacts that the one or more metabolic characteristics had on the one or more physiological characteristics. The wearable application 1120 may be configured as or otherwise support a means for transmitting a signal to a user device to cause the user device to convey information to the user associated with the one or more impacts.

The wearable application 1120 may include an application (e.g., “app”), program, software, or other component which is configured to facilitate communications with a ring 104, server 110, other user devices 106, and the like. For example, the wearable application 1120 may include an application executable on a user device 106 which is configured to receive data (e.g., physiological data) from a ring 104, perform processing operations on the received data, transmit and receive data with the servers 110, and cause presentation of data to a user 102.

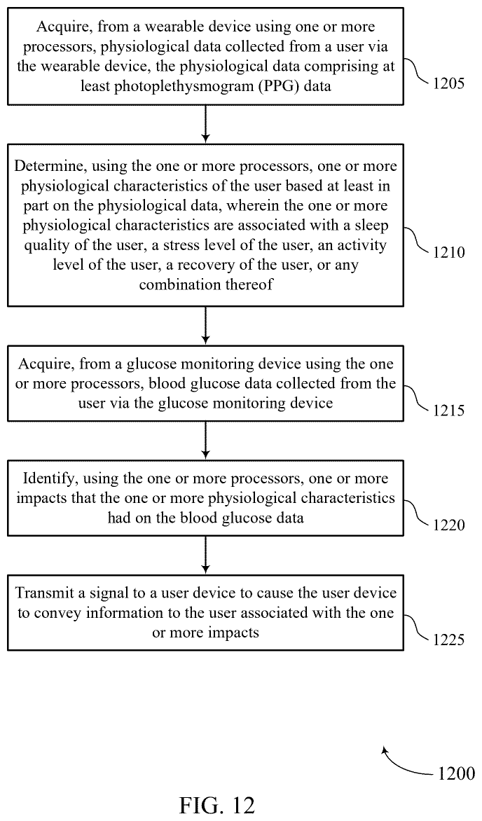

FIG. 12 shows a flowchart illustrating a method 1200 that supports techniques for evaluating impacts between physiological data and metabolic health in accordance with aspects of the present disclosure. The operations of the method 1200 may be implemented by a user device or its components as described herein. For example, the operations of the method 1200 may be performed by a user device as described with reference to FIGS. 1 through 11. In some examples, a user device may execute a set of instructions to control the functional elements of the user device to perform the described functions. Additionally, or alternatively, the user device may perform aspects of the described functions using special-purpose hardware.

At 1205, the method may include acquiring, from a wearable device using one or more processors, physiological data collected from a user via the wearable device, the physiological data comprising at least PPG data. The operations of 1205 may be performed in accordance with examples as disclosed herein. In some examples, aspects of the operations of 1205 may be performed by a physiological data manager 1025 as described with reference to FIG. 10.

At 1210, the method may include determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof. The operations of 1210 may be performed in accordance with examples as disclosed herein. In some examples, aspects of the operations of 1210 may be performed by a physiological characteristic manager 1030 as described with reference to FIG. 10.

At 1215, the method may include acquiring, from a glucose monitoring device using the one or more processors, blood glucose data collected from the user via the glucose monitoring device. The operations of 1215 may be performed in accordance with examples as disclosed herein. In some examples, aspects of the operations of 1215 may be performed by a blood glucose data manager 1035 as described with reference to FIG. 10.

At 1220, the method may include identifying, using the one or more processors, one or more impacts that the one or more physiological characteristics had on the blood glucose data. The operations of 1220 may be performed in accordance with examples as disclosed herein. In some examples, aspects of the operations of 1220 may be performed by a metabolic impact manager 1040 as described with reference to FIG. 10.

At 1225, the method may include transmitting a signal to a user device to cause the user device to convey information to the user associated with the one or more impacts. The operations of 1225 may be performed in accordance with examples as disclosed herein. In some examples, aspects of the operations of 1225 may be performed by a user device manager 1045 as described with reference to FIG. 10.

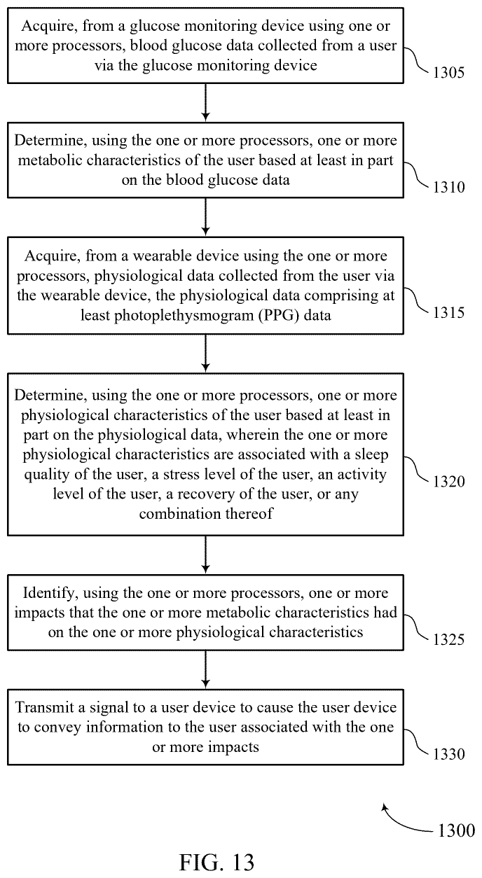

FIG. 13 shows a flowchart illustrating a method 1300 that supports techniques for evaluating impacts between physiological data and metabolic health in accordance with aspects of the present disclosure. The operations of the method 1300 may be implemented by a user device or its components as described herein. For example, the operations of the method 1300 may be performed by a user device as described with reference to FIGS. 1 through 11. In some examples, a user device may execute a set of instructions to control the functional elements of the user device to perform the described functions. Additionally, or alternatively, the user device may perform aspects of the described functions using special-purpose hardware.

At 1305, the method may include acquiring, from a glucose monitoring device using one or more processors, blood glucose data collected from a user via the glucose monitoring device. The operations of 1305 may be performed in accordance with examples as disclosed herein. In some examples, aspects of the operations of 1305 may be performed by a blood glucose data manager 1035 as described with reference to FIG. 10.

At 1310, the method may include determining, using the one or more processors, one or more metabolic characteristics of the user based at least in part on the blood glucose data. The operations of 1310 may be performed in accordance with examples as disclosed herein. In some examples, aspects of the operations of 1310 may be performed by a metabolic characteristic manager 1050 as described with reference to FIG. 10.

At 1315, the method may include acquiring, from a wearable device using the one or more processors, physiological data collected from the user via the wearable device, the physiological data comprising at least PPG data. The operations of 1315 may be performed in accordance with examples as disclosed herein. In some examples, aspects of the operations of 1315 may be performed by a physiological data manager 1025 as described with reference to FIG. 10.

At 1320, the method may include determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof. The operations of 1320 may be performed in accordance with examples as disclosed herein. In some examples, aspects of the operations of 1320 may be performed by a physiological characteristic manager 1030 as described with reference to FIG. 10.

At 1325, the method may include identifying, using the one or more processors, one or more impacts that the one or more metabolic characteristics had on the one or more physiological characteristics. The operations of 1325 may be performed in accordance with examples as disclosed herein. In some examples, aspects of the operations of 1325 may be performed by a metabolic impact manager 1040 as described with reference to FIG. 10.

At 1330, the method may include transmitting a signal to a user device to cause the user device to convey information to the user associated with the one or more impacts. The operations of 1330 may be performed in accordance with examples as disclosed herein. In some examples, aspects of the operations of 1330 may be performed by a user device manager 1045 as described with reference to FIG. 10.

It should be noted that the methods described above describe possible implementations, and that the operations and the steps may be rearranged or otherwise modified and that other implementations are possible. Furthermore, aspects from two or more of the methods may be combined.

A method for evaluating metabolic health by an apparatus is described. The method may include acquiring, from a wearable device using one or more processors, physiological data collected from a user via the wearable device, the physiological data comprising at least PPG data, determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof, acquiring, from a glucose monitoring device using the one or more processors, blood glucose data collected from the user via the glucose monitoring device, identifying, using the one or more processors, one or more impacts that the one or more physiological characteristics had on the blood glucose data, and transmitting a signal to a user device to cause the user device to convey information to the user associated with the one or more impacts.

An apparatus for evaluating metabolic health is described. The apparatus may include one or more memories storing processor executable code, and one or more processors coupled with the one or more memories. The one or more processors may individually or collectively be operable to execute the code to cause the apparatus to acquire, from a wearable device using one or more processors, physiological data collected from a user via the wearable device, the physiological data comprising at least PPG data, determine, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof, acquire, from a glucose monitoring device using the one or more processors, blood glucose data collected from the user via the glucose monitoring device, identify, using the one or more processors, one or more impacts that the one or more physiological characteristics had on the blood glucose data, and transmit a signal to a user device to cause the user device to convey information to the user associated with the one or more impacts.

Another apparatus for evaluating metabolic health is described. The apparatus may include means for acquiring, from a wearable device using one or more processors, physiological data collected from a user via the wearable device, the physiological data comprising at least PPG data, means for determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof, means for acquiring, from a glucose monitoring device using the one or more processors, blood glucose data collected from the user via the glucose monitoring device, means for identifying, using the one or more processors, one or more impacts that the one or more physiological characteristics had on the blood glucose data, and means for transmitting a signal to a user device to cause the user device to convey information to the user associated with the one or more impacts.

A non-transitory computer-readable medium storing code for evaluating metabolic health is described. The code may include instructions executable by one or more processors to acquire, from a wearable device using one or more processors, physiological data collected from a user via the wearable device, the physiological data comprising at least PPG data, determine, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof, acquire, from a glucose monitoring device using the one or more processors, blood glucose data collected from the user via the glucose monitoring device, identify, using the one or more processors, one or more impacts that the one or more physiological characteristics had on the blood glucose data, and transmit a signal to a user device to cause the user device to convey information to the user associated with the one or more impacts.

Some examples of the method, apparatus, and non-transitory computer-readable medium described herein may further include operations, features, means, or instructions for inputting the physiological data, the one or more physiological characteristics, and the blood glucose data into a machine learning classifier, wherein the machine learning classifier may be configured to output an indication of the one or more impacts.

In some examples of the method, apparatus, and non-transitory computer-readable medium described herein, and the method, apparatuses, and non-transitory computer-readable medium may include further operations, features, means, or instructions for identifying an eating gesture, a drinking gesture, or both, based at least in part on the motion data, wherein identifying the one or more impacts may be based at least in part on identifying the eating gesture, the drinking gesture, or both.

In some examples of the method, apparatus, and non-transitory computer-readable medium described herein, the one or more physiological characteristics comprise a sleep debt of the user and the one or more impacts comprise an increase in the blood glucose data of the user.

Some examples of the method, apparatus, and non-transitory computer-readable medium described herein may further include operations, features, means, or instructions for identifying one or more changes in the blood glucose data and identifying an activity pattern of the user during a time interval preceding the one or more changes in the blood glucose data, wherein the one or more impacts may be based at least in part on a temporal relationship between the activity pattern and the one or more changes.

In some examples of the method, apparatus, and non-transitory computer-readable medium described herein, identifying the one or more impacts may include operations, features, means, or instructions for identifying a sleep period that the user may be asleep based at least in part on the physiological data and identifying one or more meals consumed by the user based at least in part on the physiological data, the blood glucose data, or both, wherein the one or more impacts may be based at least in part on a temporal relationship between the sleep period and the one or more meals.

In some examples of the method, apparatus, and non-transitory computer-readable medium described herein, the glucose monitoring device comprises a component of the wearable device.

In some examples of the method, apparatus, and non-transitory computer-readable medium described herein, the wearable device comprises a wearable ring device.

A method for evaluating metabolic health by an apparatus is described. The method may include acquiring, from a glucose monitoring device using one or more processors, blood glucose data collected from a user via the glucose monitoring device, determining, using the one or more processors, one or more metabolic characteristics of the user based at least in part on the blood glucose data, acquiring, from a wearable device using the one or more processors, physiological data collected from the user via the wearable device, the physiological data comprising at least PPG data, determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof, identifying, using the one or more processors, one or more impacts that the one or more metabolic characteristics had on the one or more physiological characteristics, and transmitting a signal to a user device to cause the user device to convey information to the user associated with the one or more impacts.

An apparatus for evaluating metabolic health is described. The apparatus may include one or more memories storing processor executable code, and one or more processors coupled with the one or more memories. The one or more processors may individually or collectively be operable to execute the code to cause the apparatus to acquire, from a glucose monitoring device using one or more processors, blood glucose data collected from a user via the glucose monitoring device, determine, using the one or more processors, one or more metabolic characteristics of the user based at least in part on the blood glucose data, acquire, from a wearable device using the one or more processors, physiological data collected from the user via the wearable device, the physiological data comprising at least PPG data, determine, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof, identify, using the one or more processors, one or more impacts that the one or more metabolic characteristics had on the one or more physiological characteristics, and transmit a signal to a user device to cause the user device to convey information to the user associated with the one or more impacts.

Another apparatus for evaluating metabolic health is described. The apparatus may include means for acquiring, from a glucose monitoring device using one or more processors, blood glucose data collected from a user via the glucose monitoring device, means for determining, using the one or more processors, one or more metabolic characteristics of the user based at least in part on the blood glucose data, means for acquiring, from a wearable device using the one or more processors, physiological data collected from the user via the wearable device, the physiological data comprising at least PPG data, means for determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof, means for identifying, using the one or more processors, one or more impacts that the one or more metabolic characteristics had on the one or more physiological characteristics, and means for transmitting a signal to a user device to cause the user device to convey information to the user associated with the one or more impacts.

A non-transitory computer-readable medium storing code for evaluating metabolic health is described. The code may include instructions executable by one or more processors to acquire, from a glucose monitoring device using one or more processors, blood glucose data collected from a user via the glucose monitoring device, determine, using the one or more processors, one or more metabolic characteristics of the user based at least in part on the blood glucose data, acquire, from a wearable device using the one or more processors, physiological data collected from the user via the wearable device, the physiological data comprising at least PPG data, determine, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof, identify, using the one or more processors, one or more impacts that the one or more metabolic characteristics had on the one or more physiological characteristics, and transmit a signal to a user device to cause the user device to convey information to the user associated with the one or more impacts.

Some examples of the method, apparatus, and non-transitory computer-readable medium described herein may further include operations, features, means, or instructions for inputting the physiological data, the blood glucose data, the one or more physiological characteristics, and the one or more metabolic characteristics into a machine learning classifier, wherein the machine learning classifier may be configured to output an indication of the one or more impacts.

In some examples of the method, apparatus, and non-transitory computer-readable medium described herein, and the method, apparatuses, and non-transitory computer-readable medium may include further operations, features, means, or instructions for identifying an eating gesture, a drinking gesture, or both, based at least in part on the motion data, wherein identifying the one or more impacts may be based at least in part on identifying the eating gesture, the drinking gesture, or both.

Some examples of the method, apparatus, and non-transitory computer-readable medium described herein may further include operations, features, means, or instructions for identifying one or more changes in the blood glucose data and identifying an activity pattern of the user during a time interval preceding the one or more changes in the blood glucose data, wherein the one or more impacts may be based at least in part on a temporal relationship between the activity pattern and the one or more changes.

Some examples of the method, apparatus, and non-transitory computer-readable medium described herein may further include operations, features, means, or instructions for identifying a sleep period that the user may be asleep based at least in part on the physiological data and identifying one or more meals consumed by the user based at least in part on the physiological data, the blood glucose data, or both, wherein the one or more impacts may be based at least in part on a temporal relationship between the sleep period and the one or more meals.

In some examples of the method, apparatus, and non-transitory computer-readable medium described herein, the glucose monitoring device comprises a component of the wearable device.

In some examples of the method, apparatus, and non-transitory computer-readable medium described herein, the wearable device comprises a wearable ring device.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A system for evaluating metabolic health, comprising:
a wearable device comprising one or more sensors configured to acquire physiological data from a user, the physiological data comprising at least photoplethysmogram (PPG) data;
a glucose monitoring device configured to acquire blood glucose data from the user;
a user device communicatively coupled with the wearable device, the glucose monitoring device, or both; and
one or more processors communicatively coupled with the user device, the wearable device, the glucose monitoring device, or any combination thereof, wherein the one or more processors are configured to:
acquire the physiological data from the wearable device;
determine one or more physiological characteristics of the user based at least in part on the physiological data, wherein the one or more physiological characteristics are associated with a sleep quality of the user, a stress level of the user, an activity level of the user, a recovery of the user, or any combination thereof;
acquire the blood glucose data from the glucose monitoring device;
identify one or more impacts that the one or more physiological characteristics had on the blood glucose data; and
transmit a signal to the user device to cause the user device to convey information to the user associated with the one or more impacts.

2. The system of claim 1, wherein the one or more processors are further configured to:
transmit an additional signal to the wearable device, the glucose monitoring device, or both, based at least in part on the one or more impacts, wherein the additional signal is configured to adjust one or more operational parameters of the wearable device, the glucose monitoring device, or both; and
acquire additional physiological data via the wearable device, additional blood glucose data via the glucose monitoring device, or both, wherein the additional physiological data, the additional blood glucose data, or both, are acquired in accordance with one or more modified operational parameters based at least in part on the additional signal.

3. The system of claim 2, wherein the one or more operational parameters comprise a periodicity of measurements performed by the wearable device or the glucose monitoring device, a type of measurement performed by the wearable device, a light intensity associated with a light-emitting diode (LED) used by the wearable device to collect the physiological data, or any combination thereof.

4. The system of claim 1, wherein, to identify the one or more impacts, the one or more processors are further configured to:
input the physiological data, the one or more physiological characteristics, and the blood glucose data into a machine learning classifier, wherein the machine learning classifier is configured to output an indication of the one or more impacts.

5. The system of claim 1, wherein the physiological data further comprises motion data, wherein the one or more processors are further configured to:
identify an eating gesture, a drinking gesture, or both, based at least in part on the motion data, wherein identifying the one or more impacts is based at least in part on identifying the eating gesture, the drinking gesture, or both.

6. The system of claim 1, wherein the one or more physiological characteristics comprise a sleep debt of the user, wherein the one or more impacts comprise an increase in the blood glucose data of the user.

7. The system of claim 1, wherein, to identify the one or more impacts, the one or more processors are further configured to:
identify one or more changes in the blood glucose data; and
identify an activity pattern of the user during a time interval preceding the one or more changes in the blood glucose data, wherein the one or more impacts are based at least in part on a temporal relationship between the activity pattern and the one or more changes.

8. The system of claim 1, wherein, to identify the one or more impacts, the one or more processors are further configured to:
identify a sleep period that the user is asleep based at least in part on the physiological data; and
identify one or more meals consumed by the user based at least in part on the physiological data, the blood glucose data, or both, wherein the one or more impacts are based at least in part on a temporal relationship between the sleep period and the one or more meals.

9. The system of claim 1, wherein the glucose monitoring device comprises a component of the wearable device.

10. The system of claim 1, wherein the wearable device comprises a wearable ring device.

11. A system for evaluating metabolic health, comprising:
a wearable device comprising one or more sensors configured to acquire physiological data from a user, the physiological data comprising at least photoplethysmogram (PPG) data;
a glucose monitoring device configured to acquire blood glucose data from the user;
a user device communicatively coupled with the wearable device, the glucose monitoring device, or both; and
one or more processors communicatively coupled with the user device, the wearable device, the glucose monitoring device, or any combination thereof, wherein the one or more processors are configured to:
acquire the blood glucose data from the glucose monitoring device;
determine one or more metabolic characteristics of the user based at least in part on the blood glucose data;
acquire the physiological data from the wearable device;
determine one or more physiological characteristics of the user based at least in part on the physiological data, wherein the one or more physiological characteristics are associated with a sleep quality of the user, a stress level of the user, an activity level of the user, a recovery of the user, or any combination thereof;
identify one or more impacts that the one or more metabolic characteristics had on the one or more physiological characteristics; and
transmit a signal to the user device to cause the user device to convey information to the user associated with the one or more impacts.

12. The system of claim 11, wherein the one or more processors are further configured to:
transmit an additional signal to the wearable device, the glucose monitoring device, or both, based at least in part on the one or more impacts, wherein the additional signal is configured to adjust one or more operational parameters of the wearable device, the glucose monitoring device, or both; and
acquire additional physiological data via the wearable device, additional blood glucose data via the glucose monitoring device, or both, wherein the additional physiological data, the additional blood glucose data, or both, are acquired in accordance with one or more modified operational parameters based at least in part on the additional signal.

13. The system of claim 11, wherein, to identify the one or more impacts, the one or more processors are further configured to:
input the physiological data, the blood glucose data, the one or more physiological characteristics, and the one or more metabolic characteristics into a machine learning classifier, wherein the machine learning classifier is configured to output an indication of the one or more impacts.

14. The system of claim 11, wherein the physiological data further comprises motion data, wherein the one or more processors are further configured to:
identify an eating gesture, a drinking gesture, or both, based at least in part on the motion data, wherein identifying the one or more impacts is based at least in part on identifying the eating gesture, the drinking gesture, or both.

15. The system of claim 11, wherein, to identify the one or more impacts, the one or more processors are further configured to:
identify one or more changes in the blood glucose data; and
identify an activity pattern of the user during a time interval preceding the one or more changes in the blood glucose data, wherein the one or more impacts are based at least in part on a temporal relationship between the activity pattern and the one or more changes.

16. The system of claim 11, wherein, to identify the one or more impacts, the one or more processors are further configured to:
identify a sleep period that the user is asleep based at least in part on the physiological data; and
identify one or more meals consumed by the user based at least in part on the physiological data, the blood glucose data, or both, wherein the one or more impacts are based at least in part on a temporal relationship between the sleep period and the one or more meals.

17. The system of claim 11, wherein the glucose monitoring device comprises a component of the wearable device.

18. The system of claim 11, wherein the wearable device comprises a wearable ring device.

19. A method for evaluating metabolic health, comprising:
acquiring, from a wearable device using one or more processors, physiological data collected from a user via the wearable device, the physiological data comprising at least photoplethysmogram (PPG) data;
determining, using the one or more processors, one or more physiological characteristics of the user based at least in part on the physiological data, wherein the one or more physiological characteristics are associated with a sleep quality of the user, a stress level of the user, an activity level of the user, a recovery of the user, or any combination thereof;
acquiring, from a glucose monitoring device using the one or more processors, blood glucose data collected from the user via the glucose monitoring device;
identifying, using the one or more processors, one or more impacts that the one or more physiological characteristics had on the blood glucose data; and
transmitting a signal to a user device to cause the user device to convey information to the user associated with the one or more impacts.

20. The method of claim 19, further comprising:
transmitting an additional signal to the wearable device, the glucose monitoring device, or both, based at least in part on the one or more impacts, wherein the additional signal is configured to adjust one or more operational parameters of the wearable device, the glucose monitoring device, or both; and
acquiring additional physiological data via the wearable device, additional blood glucose data via the glucose monitoring device, or both, wherein the additional physiological data, the additional blood glucose data, or both, are acquired in accordance with one or more modified operational parameters based at least in part on the additional signal.
